(19) United States
(12) Patent Application Publication
(10) Pub. No.: US 2025/0009803 A1
(43) Pub. Date: Jan. 9, 2025

(54) TREATMENT OF KIDNEY DISEASE

(71) Applicants: Timothy A. BERTRAM, George Town (KY); Deepak JAIN, Winston-Salem, NC (US)

(72) Inventors: Timothy A. BERTRAM, George Town (KY); Deepak JAIN, Winston-Salem, NC (US)

(21) Appl. No.: 18/699,849

(22) PCT Filed: Oct. 14, 2022
(86) PCT No.: PCT/US2022/078153

§ 371 Date: Apr. 9, 2024

Related U.S. Application Data
(60) Provisional application No. 63/255,885, filed on Oct. 14, 2021.
(60) Provisional application No. 63/307,801, filed on Feb. 8, 2022.
(60) Provisional application No. 63/321,204, filed on Mar. 18, 2022.

(51) Int. Cl. A61K 35/22; A61P 13/12

(52) U.S. Cl. CPC A61K 35/22; A61P 13/12

(57) ABSTRACT

Use of an autologous e.g., selected, renal cell therapeutic for improving kidney function of a human patient having diabetic chronic kidney disease is provided herein. The use of the therapeutic, administered in first and second injections, increases patient kidney function. e.g., estimated glomerular filtration rate, over the course of at least one year and stabilizes other clinical parameters that decline during kidney disease.

### CROSS-REFERENCE TO RELATED APPLICATIONS

This application claims benefit of priority to U.S. Provisional Application Ser. No. 63/255,885 filed Oct. 14, 2021, U.S. Provisional Application Ser. No. 63/307,801 filed Feb. 8, 2022, and U.S. Provisional Application Ser. No. 63/321,204 filed Mar. 18, 2022, the three of which are entitled “TREATMENT OF KIDNEY DISEASE” and are herein incorporated by reference in their entireties.

### BACKGROUND

Chronic kidney disease (CKD) is characterized by progressive nephropathy that, without therapeutic intervention, will worsen; ultimately the patient may reach end stage renal disease (ESRD). CKD is often accompanied by adverse outcomes owing to underlying comorbidities and/or risk factors including hypertension and renovascular disease (Khan et al., 2002; Stenvinkel P. Chronic kidney disease—a public health priority and harbinger of premature cardiovascular disease J Intern med. 2010; 268:456-67). Due to serious comorbidities, patients with CKD are 5-11 times more likely to suffer premature death than survive to progress to end stage renal disease (ESRD) (Collins et al., 2003; Smith et al., 2004).

In diabetic nephropathy, the pathomorphological sequence of nephron loss with decapitation of the glomerulus and progressive tubular fibrosis has been well described (Lowen J, Grone E F, Gross-Weissmann M L, et al. Pathomorphological sequence of nephron loss in diabetic nephropathy. Am J Physiol Renal Physiol 2021; 321: F600-F616). As in other causes of CKD, once nephron loss has begun, no repair of glomeruli can occur. Most standard of care treatments for CKD are small molecules targeting biochemical pathways in the kidney to affect single or related co-morbidities, but the underlying glomerular and tubulointerstitial dysfunction remains unaltered as new nephrons cannot be generated. Ultimately, in patients that progress to ESRD, renal replacement therapy (dialysis or transplantation) is required for survival.

There is a healthcare need for CKD treatment options that improve function of diseased kidneys. Improving the function of diseased kidneys may not only improve filtration of the blood, the kidney's primary function, but also delay or prevent the need for renal replacement therapy and/or alleviate co-morbidities associated with CKD. Renal Autologous Cell Therapy (REACT) is a therapeutic composition including an autologous renal cell, e.g., selected renal cell (SRC), population for injection into the renal cortex of a human CKD patient. Administration of two injections of REACT to CKD patients has now been demonstrated to improve CKD patient kidney function, e.g., at least in part by consistently increasing their estimated glomerular filtration rate (eGFR) over time, including over at least one year.

### BRIEF SUMMARY

The present disclosure describes a method of improving kidney function to a human patient having diabetic chronic kidney disease (D-CKD). In the method, first and second injections of a therapeutic composition that includes a selected renal cell (SRC) population are administered into the renal cortex of at least one kidney of the human patient. The first and second injections of the therapeutic composition are administered between approximately 3 and 12 months apart. The therapeutic composition includes about 1.0-9.0×10⁶ cells of the SRC population per gram estimated kidney weight. The SRC population includes bioactive renal cells prepared from cultured renal cells of a kidney biopsy of the patient. The administration of the first and second injections thereby improves kidney function of the human patient.

The present disclosure describes yet another method of improving kidney function to a human patient having D-CKD. In the method, a first injection of a therapeutic composition including a SRC population is administered into the renal cortex of at least one kidney of the human patient. A second injection of the therapeutic composition is administered into the renal cortex of at least one kidney of the human patient if, approximately 3 to 18 months after the first injection, a triggering event is detected. The triggering event may be a reduction, or insignificant change, in patient eGFR relative to patient baseline eGFR or it may be an increase, or insignificant change, in patient uACR relative to patient baseline uACR. The second injection is administered within approximately 30 days after detecting the triggering event. The therapeutic composition includes about 1.0-9.0×10⁶ cells of the SRC population per gram estimated kidney weight. The SRC population includes bioactive renal cells prepared from cultured renal cells of a kidney biopsy of the patient. The administration of the first and second injections thereby improves kidney function of the human patient.

### BRIEF DESCRIPTION OF FIGURES

FIG. 1: Provides data showing change in eGFR in advanced Type 2 D-CKD patients having received two REACT, e.g., SRC-containing, injections. Change in eGFR is shown for the patient group as a whole and, separately, for patients categorized as low responders and patients categorized as moderate/high responders to treatment (p=0.768, ANOVA).

FIG. 2A-B: Show response of eGFR (eGFR based on 2009 CKD-EPI using sCr only in FIG. 2A; eGFR based on 2012 CKD-EPI using creatinine and cystatin C in FIG. 2B) to SRCs injection in D-CKD patients having received two REACT, e.g., SRC-containing, injections. Day 0 represents the date of the first injection.

FIG. 3A-C: Provides results of an interim analysis of primary clinical renal function markers, estimated glomerular filtration (eGFR; top solid and broken line for REACT-treated patients and bottom solid and broken line for SOC-treated patients; FIGS. 3A and 3C) and albuminuria (urine albumin-to-creatinine ratio (UACR); FIG. 3B), in D-CKD patients treated with two REACT, e.g., SRC-containing, injections versus standard of care (SOC).

FIG. 4: Provides data comparing change in eGFR in D-CKD patients having received two REACT, e.g., SRC-containing, injections versus D-CKD patients having been treated with SOC. Top solid and broken line, REACT-treated patients; bottom solid and broken line, SOC-treated patients. P-values were calculated using two sided welch two sample T test.

FIG. 5A-F: Provides data showing stabilization in urinary albumin/creatinine (UAUC; FIG. 5A), hemoglobin (FIG. 5B), serum phosphate (FIG. 5C), serum potassium (FIG. 5D), blood pressure (FIG. 5E) and intact parathyroid hormone (iPTH; FIG. 5F) in a D-CKD subset of patients evaluated with FACS and having received two REACT, e.g., SRC-containing, injections. Stabilization responses shown for the patient group as a whole and, separately, for patients categorized as low responders and patients categorized as moderate/high responders.

FIG. 6A-C: Provides results of an interim analysis of hemoglobin (FIG. 6A), serum calcium (FIG. 6B) and serum phosphorus (FIG. 6C) in D-CKD patients treated with two REACT, e.g., SRC-containing, injections versus SOC. Light colored bars, REACT-treated. Dark colored bars, SOC-treated.

FIG. 7: Shows response of eGFR (CKD-EPI Creatinine 2009 Equation) in late stage IV D-CKD having received two REACT, e.g., SRC-containing, injections. Linear Mixed Effects Model: eGFR pre- and post-injection with cohort (dashed line) and individual patient slopes (solid lines). Slope deceleration occurs at first injection, Time=0. Slope decelerates, e.g., improves, from −6.5 mL/min/1.73 m² per year pre-injection to −3.9 ml mL/min/1.73 m² post injection.

FIG. 8: Provides, for late stage IV D-CKD patients, a graph showing extrapolated pre-injection eGFR slope (−6.5 ml/min/1.73 m²) from the time of first injection and estimated time to dialysis (approximately 10.1 months) without interventions assuming a dialysis initiation at an eGFR of 10 mL/min/1.73 m²; calculated eGFR slope improvement post first SRC-containing injection (2.6 ml/min/1.73 m²) extended estimated time to dialysis to 30 months.

### DETAILED DESCRIPTION

Described herein are methods of improving kidney function to a human patient having diabetic chronic kidney disease (D-CKD). In D-CKD, the CKD may be a complication of type 1 or type 2 diabetes resulting from high blood sugar levels that, over time, damage blood vessels and nephrons in the kidneys. The damage to the kidneys reduces their function, resulting in the kidney disease. Furthermore, because diabetes damages small blood vessels, not just those in the kidney, the diabetes may also cause hypertension. Hypertension involvement in D-CKD patients accelerates the CKD. Thus, in the methods of improving kidney function of a human patient having D-CKD, the patient may be a D-CKD patient having type 1 diabetes or a D-CKD patient having type 2 diabetes. In the methods of improving kidney function, the patient may be a D-CKD patient having type 1 diabetes or type 2 diabetes and/or may have hypertension, e.g., high blood pressure.

In the methods of improving the kidney function to a human patient having D-CKD, the CKD of the patient may be classified as being in stage I CKD, stage II CKD, stage III CKD or stage IV CKD. If the patient having D-CKD is classified as being in stage I CKD, the patient may have an eGFR of greater than approximately 90 mL/min/1.73 m² but have signs of kidney damage determined by detecting higher than normal levels of protein, e.g., albumin, in blood, by detecting higher than normal levels of urea in urine, detecting higher than normal levels of creatinine, or by detecting structural damage using imaging techniques, e.g., CT scan, magnetic resonance imaging, ultrasound or x-ray with contrast. If the patient having D-CKD is classified as being in stage II CKD, the patient may have an eGFR of approximately 60 to 89 mL/min/1.73 m². If the patient having D-CKD is classified as being in stage III CKD, the patient may have an eGFR of approximately 30 to 59 mL/min/1.73 m². If the patient having D-CKD is classified as being in stage IV CKD, the patient may have an eGFR of approximately 15 to 29 mL/min/1.73 m². The patient having D-CKD, treated by the methods, may have an eGFR of approximately 15-90, 15-75, 15-60, 15-45, 15-30, 15-20, 14-20, 20-90, 20-75, 20-60, 20-45, 20-30, 30-90, 30-75, 30-60, 30-45, 40-90, 40-75, 40-60, or 50-90 mL/min//1.73 m². The patient having D-CKD, treated by the methods, may have an eGFR of less than approximately 20 mL/min/1.73 m², or may have an eGFR of between about 20 mL/min/1.73 m² and about 50 mL/min/1.73 m², or between about 20 mL/min/1.73 m² and about 60 mL/min/1.73 m², or between about 20 mL/min/1.73 m² and about 90 mL/min/1.73 m².

In the methods of improving kidney function of the human patient having D-CKD, the improvement in kidney function may be, or include, an increase in eGFR of the patient or a stabilization or reduction of uACR of the patient. In the methods of improving kidney function of the human patient having D-CKD, the improvement in kidney function may be, or include, a restoration in regulation of: erythropoiesis by the kidney, blood or serum potassium level by the kidney, blood or serum phosphorus level by the kidney, and/or blood or serum calcium of the kidney. In the methods of improving kidney function of the human patient having D-CKD, the improvement in kidney function may be, or include, increased blood filtration, e.g., that may be determined as a stabilization (reduction in rate of decline) or increase in eGFR, or stabilization of uACR, by the kidney.

In the method of improving kidney function of the human patient having D-CKD, if the improving kidney function of the patient increases the patient's eGFR, the increase in the patient's eGFR may be an increase of about 1-10 ml/min/1.73 m², about 1-9 ml/min/1.73 m², about 1-8 ml/min/1.73 m², about 1-7 ml/min/1.73 m², about 1-6 ml/min/1.73 m², about 1-5 ml/min/1.73 m², about 1-4 ml/min/1.73 m², about 1-3 ml/min/1.73 m² or about 3-5 ml/min/1.73 m². The increase may be relative to the patient's eGFR approximately one month from administration of the first or second injection with the therapeutic composition. The increase in the patient's eGFR may be an increase of at least about 1 ml/min/1.73 m², at least about 2 ml/min/1.73 m², at least about 3 ml/min/1.73 m², at least about 4 ml/min/1.73 m², or at least about 5 ml/min/1.73 m². The increase may be relative to the patient's eGFR approximately one month from administration of the first or second injection with the therapeutic composition.

In the method of improving kidney function of the human patient having D-CKD, if the improving kidney function stabilizes or reduces the patient's uACR, the improving kidney function may stabilize the patient's uACR. If the improving kidney function stabilizes the patient's uACR, then the patient's uACR may be maintained at a level within about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1%. It may be maintained at the level within about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% relative to the patient's uACR approximately one month from administrations of the first or second injection with the therapeutic composition. If the improving kidney function reduces the patient's uACR, then the patient's uACR may decrease by about 1 to 20%, by about 1 to 19%, by about 1 to 18%, by about 1 to 17%, by about 1 to 16%, by about 1 to 15%, by about 1 to 14%, by about 1 to 13%, by about 1 to 12%, by about 1 to 11%, by about 1 to 10%, by about 1 to 9%, by about 1 to 8%, by about 1 to 7%, by about 1 to 6%, by about 1 to 5%, by about 5 to 20%, by about 5 to 15% or by about 10% to 20%. The reduction may be relative to the patient's uACR approximately one month from administration of the first or second injection with the therapeutic composition.

In the method of improving kidney function of the human patient having D-CKD, if the improvement in kidney function restores regulation of erythropoiesis by the kidney, the restoration of regulation of erythropoiesis by the kidney may stabilize the patient's blood hemoglobin level at, or a return of the patient's blood hemoglobin levels to, normal levels, e.g., between about 13.0 and 17.5 g/dL. If the regulation of erythropoiesis stabilizes the patient's blood hemoglobin levels, the stabilization may maintain the patient's blood hemoglobin at a level of between about 13.0 and 17.5 g/dL. It will be understood that the maintaining the patient's blood hemoglobin at a level of between about 13.0 and 17.5 g/dL does not require that the patient's blood hemoglobin level be static over time. Rather, it will be understood that the maintaining the patient's blood hemoglobin at a level of between about 13.0 and 17.5 g/dL permits changes in the patient's blood hemoglobin level, e.g., of less than about 0.5 g/dL, by less than about 1.0 g/dL, by less than about 1.5 g/dL or by less than about 2.0 g/dL, so long as the changes do not result in the patient's blood hemoglobin level falling outside the about 13.0 to 17.5 g/dL range. If the improvement in kidney function restores regulation of erythropoiesis by the kidney, and if the D-CKD patient's blood hemoglobin level is less than about 13.0 g/dL, then the restoration of regulation of erythropoiesis may increase the patient's blood hemoglobin to at least about 13.0 g/dL and then maintain the patient's blood hemoglobin level at between about 13.0 and 17.5 g/dL. If the improvement in kidney function restores regulation of erythropoiesis by the kidney, and if the D-CKD patient's blood hemoglobin level is less than about 13.0 g/dL, then the restoration of regulation of erythropoiesis may increase the patient's blood hemoglobin to at least about 13.0 g/dL, e.g., by increasing the patient's blood hemoglobin level by at least about 0.5 g/dL, at least about 1.0 g/dL, at least about 1.5 g/dL or at least about 2/0 g/dL, and then once between about 13.0 and 17.5 g/dL, maintains the patient's blood hemoglobin at a level between about 13.0 and 17.5 g/dL.

In the methods of improving kidney function of the human patient having D-CKD, if the improving kidney function restores regulation of blood or serum potassium by the patient's kidney, and the D-CKD patient's blood or serum potassium level is between about 3.5 and 5.0 mmol/L, then the restoring regulation of the patient's blood or serum potassium level by the kidney may maintain the patient's blood or serum potassium at a level of between about 3.5 and 5.0 mmol/L. It will be understood that the maintaining the patient's blood or serum potassium at a level of between about 3.5 and 5.0 mmol/L does not require that the patient's blood or serum potassium level be static over time. Rather, it will be understood that the maintaining the patient's blood or serum potassium at a level of between about 3.5 and 5.0 mmol/L permits changes in the patient's blood or serum potassium level, e.g., by less than about 0.5 mmol/L, by less than about 1.0 mmol/L, by less than about 1.5 mmol/L or by less than about 2.0 mmol/L, so long as the changes do not result in the patient's blood or serum potassium level falling outside the about 3.5 and 5.0 mmol/L range. If the improvement in kidney function restores regulation of blood or serum potassium by the patient's kidney, and if the D-CKD patient's blood or serum potassium level is greater than about 5.0 mmol/L, then the restoration of regulation of blood or serum potassium by the patient's kidney may be a regulation that decreases the patient's blood or serum potassium level to at least about 5.0 mmol/L and then maintain the patient's blood or serum potassium at levels between about 3.5 and 5.0 mmol/L. If the improvement in kidney function restores regulation of blood or serum potassium by the patient's kidney, and if the D-CKD patient's blood or serum potassium level is greater than about 5.0 mmol/L, then the restoration of regulation of blood or serum potassium by the patient's kidney may be a regulation that decreases the patient's blood or serum potassium level to at least about 5.0 mmol/L, e.g., by decreasing the patient's blood or serum potassium level by at least about 0.5 mmol/L, at least about 1.0 mmol/L, at least about 1.5 mmol/L or at least about 2.0 mmol/L, and then once between about 3.5 and 5.0 mmol/L, maintains the patient's blood or serum potassium at levels between about 3.5 and 5.0 mmol/L.

In the methods of improving kidney function of the human patient having D-CKD, if the improving kidney function restores regulation of blood or serum phosphorus by the patient's kidney, and the D-CKD patient's blood or serum phosphorus level is between about 3.5 and 5.5 mg/dL, then the restoring regulation of the patient's blood or serum phosphorus level by the kidney may be a regulation that maintains the patient's blood or serum phosphorus at a level of between about 3.5 and 5.5 mg/dL. It will be understood that the maintaining the patient's blood or serum phosphorus at a level of between about 3.5 and 5.5 mg/dL does not require that the patient's blood or serum phosphorus level be static over time. Rather, it will be understood that the maintaining the patient's blood or serum phosphorus at a level of between about 3.5 and 5.5 mg/dL permits changes in the patient's blood or serum phosphorus level, e.g., by less than about 0.5 mg/dL, by less than about 1.0 mg/dL, by less than about 1.5 mg/dL or by less than about 2.0 mg/dL, so long as the changes do not result in the patient's blood or serum phosphorus level falling outside the about 3.5 and 5.5 mg/dL range. If the improvement in kidney function restores regulation of blood or serum phosphorus by the patient's kidney, and if the D-CKD patient's blood or serum phosphorus level is greater than about 5.5 mg/dL, then the restoration of regulation of blood or serum phosphorus by the patient's kidney may be a regulation that decreases the patient's blood or serum phosphorus level to at least about 5.5 mg/dl and then maintains the patient's blood or serum phosphorus at levels between about 3.5 and 5.5 mg/dL. If the improvement in kidney function restores regulation of blood or serum phosphorus by the patient's kidney, and if the D-CKD patient's blood or serum phosphorus level is greater than about 5.5 mg/dL, then the restoration of regulation of blood or serum phosphorus by the patient's kidney may be a regulation that decreases the patient's blood or serum phosphorus level to at least about 5.5 mg/dL, e.g., by decreasing the patient's blood or serum phosphorus level by at least about 0.5 mg/dL, at least about 1.0 mg/dL, at least about 1.5 mg/dL or at least about 2.0 mg/dL, and then once between about 3.5 and 5.5 mg/dL, maintains the patient's blood or serum phosphorus at levels between about 3.5 and 5.5 mg/dL.

In the methods of improving kidney function of the human patient having D-CKD, if the improving kidney function restores regulation of blood or serum calcium by the patient's kidney, and the D-CKD patient's blood or serum calcium level is between about 8.5 and 10.5 mg/dl, then the restoring regulation of blood or serum calcium level by the patient's kidney may be a regulation that maintains the patient's blood or serum calcium at a level of between about 8.5 and 10.5 mg/dL. It will be understood that the maintaining the patient's blood or serum calcium at a level of between about 8.5 and 10.5 mg/dL does not require that the patient's blood or serum calcium level be static over time. Rather, it will be understood that the maintaining the patient's blood or serum calcium at a level of between about 8.5 and 10.5 mg/dL permits changes in the patient's blood or serum calcium level, e.g., by less than about 0.5 mg/dL, by less than about 1.0 mg/dL, by less than about 1.5 mg/dL or by less than about 2.0 mg/dL, so long as the changes do not result in the patient's blood or serum calcium level falling outside the about 8.5 and 10.5 mg/dL range. If the improvement in kidney function restores regulation of blood or serum calcium by the patient's kidney, and if the D-CKD patient's blood or serum calcium level is less than about 8.5 mg/dL, then the restoration of regulation of blood or serum calcium by the patient's kidney may be a regulation that increases the patient's blood or serum calcium level to at least about 8.5 mg/dL and then maintains the patient's blood or serum calcium at levels between about 8.5 and 10.5 mg/dL. If the improvement in kidney function restores regulation of blood or serum calcium by the patient's kidney, and if the D-CKD patient's blood or serum calcium level is less than about 8.5 mg/dL, then the restoration of regulation of blood or serum calcium by the patient's kidney may be a regulation that increases the patient's blood or serum calcium level to at least about 8.5 mg/dL, e.g., by increasing the patient's blood or serum calcium level by at least about 0.5 mg/dL, at least about 1.0 mg/dL, at least about 1.5 mg/dL or at least about 2.0 mg/dL, and then once between about 8.5 and 10.5 mg/dL, maintains the patient's blood or serum calcium at levels between about 8.5 and 10.5 mg/dL

In the methods of improving kidney function of the human patient having D-CKD, if the improving kidney function increases blood filtration by the patient's kidney, the increase in filtration by the patient's kidney may be an increase of at least about 1%, at least about 2%, at least about 3%, at least about 4%, at least about 5% at least about 6%, at least about 7%, at least about 8%, at least about 9%, at least about 10%, at least about 11%, at least about 12%, at least about 13%, at least about 14% or at least about 15%. The increase may be relative to the patient's kidney blood filtration at a time within approximately 1 month from administration of the first or second injection with the therapeutic composition. In the methods of improving kidney function of the human patient having D-CKD, if the improving kidney function increases blood filtration by the patient's kidney, the increase in filtration by the patient's kidney may be an increase of between about 1% and 15%, between about 1% and 12%, between about 1% and 10%, between about 1% and 7%, between about 1% and 5%, between about 3% and 15%, between about 3% and 12%, between about 3% and 10%, between about 3% and 7%, between about 3% and 5%, 5% and 15%, between about 5% and 12%, between about 5% and 10%, or between about 5% and 7%. The increase may be relative to the patient's kidney blood filtration approximately one month from administration of the first or second injection with the therapeutic composition. Filtration by the patient's kidney may be determined, or detected, using any clinical assay or test known by those of skill in the art that measures kidney filtration. Such assays or tests include, but are not limited to, those that determine eGFR, e.g., those that include determining Cystatin-C; serum creatinine; iohexol; or nuclear scinitigraphic analysis (e.g. Tc-99 m DTPA (diethylenetriaminepentaacetic acid), and/or those that measure uACR. If the improving kidney function of the human patient having D-CKD increases blood filtration by the patient's kidney, and the patient's blood filtration increase is determined by eGFR, then the increase in kidney function may stabilize, (e.g., by reducing the patient's rate of decline in eGFR by at least about 25%, 30%, 35%, 40%, 45%, 50%, 55%, 60%, 65%, 70%, 75%, 80%, 85%, 90%, 95% or 100% eGFR) or increase (e.g., by at least about 1%, 2%, 3%, 4%, 5%, 6%, 7%, 8%, 9%, 10%, 11%, 12%, 13%, 14% or 15%) the patient's eGFR. If the improving kidney function of the human patient having D-CKD increases blood filtration of the patient's kidney, and the patient's blood filtration is determined by a reduction in the patient's rate of decline in eGFR, the patient may be a D-CKD patient in stage IV CKD or late stage IV CKD, e.g., having an eGFR of about less than 20 mL/min/1.73 m², and the reduction in rate of decline in eGFR may be by at least about 25%, 30%, 35%, 40%, 45%, 50%, 55%, 60%, 65%, 70%, 75%, 80%, 85%, 90%, 95% or 100%.

In the method of improving kidney function of the human patient having D-CKD, the improving kidney function may be or include any combination of two, or all three of: increasing eGFR of the patient, stabilizing or reducing uACR of the patient, and/or increasing blood filtration by the kidneys of the patient. In the method of improving kidney function of the human patient having D-CKD, the improving kidney function may be a restoring the regulation of any combination of two, or three, or all four of: erythropoiesis by the kidney, blood or serum potassium level by the kidney, blood or serum phosphorus level by the kidney and/or blood or serum calcium level by the kidney. In the method of improving kidney function of the human patient having D-CKD, the improving kidney function may be or include any combination of two, three, four, five, six or all seven of: restoring regulation of erythropoiesis by the kidney, restoring regulation of blood or serum potassium level by the kidney, restoring regulation of blood or serum phosphorus level by the kidney, restoring regulation of blood or serum calcium level by the kidney, increasing eGFR of the patient, stabilizing or reducing uACR of the patient, and/or increasing blood filtration by kidneys of the patient.

In any of the methods of improving kidney function of the human patient having D-CKD, the improvement in kidney function, (e.g., increased patient eGFR, stabilized or reduced patient uACR, restored regulation of erythropoiesis by the patient's kidney, restored regulation of blood or serum potassium level by the patient's kidney, restored regulation of blood or serum phosphorus level by the patient's kidney, restored regulation of blood or serum calcium level by the patient's kidney, and/or increased blood filtration (e.g., stabilization (reduction in rate of decline in eGFR) by the patient's kidney), may be an improvement measurable, or determinable, from first relative to a second time point. The first time point may be a time within approximately three months prior to, or after, administration of the first injection with the therapeutic composition. The first time point may be a time within approximately 12 weeks to 1 week, approximately 12 weeks to 2 weeks, approximately 12 weeks to 3 weeks, approximately 12 weeks to 4 weeks, approximately 8 weeks to 1 week, approximately 8 weeks to 2 weeks, approximately 8 weeks to 3 weeks or approximately 8 weeks to 4 weeks prior to, or after administration of the first injection of the therapeutic composition. The first time point may be a time within approximately one month prior to, or after, administration of the first injection with the therapeutic composition. The first time point may be a time within approximately 1 to 30 days, approximately 1 to 28 days, approximately 1 to 26 days, approximately 1 to 24 days, approximately 1 to 22 days, approximately 1 to 20 days, approximately 1 to 18 days, approximately 1 to 16 days, approximately 1 to 14 days, approximately 1 to 12 days, approximately 1 to 10 days, approximately 1 to 8 days, approximately 1 to 6 days, approximately 1 to 4 days or approximately 1 to 2 days prior to, or after, administration of the first injection of the therapeutic composition. The first time point may be within about 24 hours prior to, or after, administration of the first injection with the therapeutic composition.

Alternatively, the first time point may be a time within about three months prior to, or after, administration of the second injection of the therapeutic composition. The first time point may be a time within approximately 12 weeks to 1 week, approximately 12 weeks to 2 weeks, approximately 12 weeks to 3 weeks, approximately 12 weeks to 4 weeks, approximately 8 weeks to 1 week, approximately 8 weeks to 2 weeks, approximately 8 weeks to 3 weeks or approximately 8 weeks to 4 weeks prior to, or after administration of the second injection with the therapeutic composition. The first time point may be a time within about a month prior to, or after, administration of the second injection of the therapeutic composition. The first time point may be a time approximately 1 to 30 days, approximately 1 to 28 days, approximately 1 to 26 days, approximately 1 to 24 days, approximately 1 to 22 days, approximately 1 to 20 days, approximately 1 to 18 days, approximately 1 to 16 days, approximately 1 to 14 days, approximately 1 to 12 days, approximately 1 to 10 days, approximately 1 to 8 days, approximately 1 to 6 days, approximately 1 to 4 days or approximately 1 to 2 days prior to, or after, administration of the second injection of the therapeutic composition. The first time point may be a time within about 24 hours prior to, or after, the second injection with the therapeutic composition.

The second time point, for comparison relative to the first time point, may be a time approximately one year after administration of the first injection with the therapeutic composition. The second time point may be a time approximately 9 months, approximately 10 months, approximately 11 months, approximately 12 months, approximately 13 months, approximately 14 months, approximately 15 months, approximately 16 months, approximately 17 months, approximately 18 months, approximately 19 months, approximately 20 months, approximately 21 months, approximately 22 months, approximately 23 months or approximately 24 months after the administration of the first injection of the therapeutic composition. The second time point may be a time that is approximately 9 to 24 months, approximately 9 to 22 months, approximately 9 to 20 months, approximately 9 to 18 months, approximately 10 to 24 month, approximately 12 to 24 months, approximately 12 to 20 months or approximately 12 to 18 months after the administration of the first injection with the therapeutic composition.

The second time point, alternatively, may be a time approximately one year after administration of the second injection with the therapeutic composition. The second time point may be a time approximately 9 months, approximately 10 months, approximately 11 months, approximately 12 months, approximately 13 months, approximately 14 months, approximately 15 months, approximately 16 months, approximately 17 months, approximately 18 months, approximately 19 months, approximately 20 months, approximately 21 months, approximately 22 months, approximately 23 months or approximately 24 months after the administration of the second injection of the therapeutic composition. The second time point may be a time approximately 9 to 24 months, approximately 9 to 22 months, approximately 9 to 20 months, approximately 9 to 18 months, approximately 10 to 24 month, approximately 12 to 24 months, approximately 12 to 20 months or approximately 12 to 18 months after the administration of the second injection of the therapeutic composition.

By way of example, in the methods of improving kidney function to a human D-CKD patient in which the improvement in kidney function increases the patient's eGFR, the patient's eGFR may increase from a first time point, e.g., within about one month of administration of either the first or the second injection with the therapeutic composition, to a second time point, e.g., approximately 12 to 24 months after administration of either the first or second injection with the therapeutic composition. The patient's improvement in kidney function may increase the patient's eGFR from a first time point, e.g., within about a month of administration of the first injection with the therapeutic composition, to a second time point that may be about 12 to 24 months, e.g., approximately 12 or 18 or 24 months, after administration of the first injection with the therapeutic composition. The improvement in kidney function may increase the patient's eGFR from a first time point, e.g., within about a month of administration of the second injection with the therapeutic composition, to a second time point that may be about 12 to 24 months, e.g., approximately 12 or 18 or 24 months, after administration of the second injection with the therapeutic composition. The improvement in kidney function may increase the patient's eGFR by about 1-10 ml/min/1.73 m², about 1-5 ml/min/1.73 m², about 1-3 ml/min/1.73 m², or about 3-5 ml/min/1.73 m² from a first time point, e.g., within about month of administration of the first injection with the therapeutic composition, to a second time point that may be about 12 to 24 months, e.g., approximately 12 or 18 or 24 months, after administration of the first injection with the therapeutic composition. The improvement in kidney function may increase the patient's eGFR by about 1-10 ml/min/1.73 m², about 1-5 ml/min/1.73 m², about 1-3 ml/min/1.73 m², or about 3-5 ml/min/1.73 m² from a first time point, e.g., within about a month of administration of the second injection with the therapeutic composition, to a second time point that may be about 12 to 24 months, e.g., approximately 12 or 18 or 24 months, after administration of the second injection with the therapeutic composition. The improvement in kidney function may increase the patient's eGFR by at least about 2 ml/min/1.73 m², or at least about 3 ml/min/1.73 m², or at least about 4 ml/min/1.73 m², or at least about 5 ml/min/1.73 m² from a first time point, e.g., within about a month of administration of the first injection with the therapeutic composition, to a second time point that may be about 12 to 24 months, e.g., approximately 12 or 18 or 24 months, after administration of the first injection with the therapeutic composition. The improvement in kidney function may increase the patient's eGFR by at least about 2 ml/min/1.73 m², or at least about 3 ml/min/1.73 m², or at least about 4 ml/min/1.73 m², or at least about 5 ml/min/1.73 m² from a first time point, e.g., within about a month of administration the second injection with the therapeutic composition, to a second time point that may be about 12 to 24 months, e.g., approximately 12 or 18 or 24 months, after administration of the second injection with the therapeutic composition. The improvement in kidney function may be an increase that continues over time, e.g., in which the patient's eGFR increases from the first time point to about the midpoint between the first and the second time point, and again increases from between about the midpoint between the first and the second time point to the second time point. The increases in eGFR need not be identical. For example, the improvement in kidney function may be an increase such that the patient's eGFR increases from the first time point to about 6 months from the first time point, and again increases from about the 6 months from the first time point to the second time point if the first time point is within about a month from the administration of the first or second injection of the therapeutic composition and the second time point is about 12 months after administration of the first or second, respectively, injection of the therapeutic composition. Again the increases need not be the same, e.g., identical.

In the method of improving kidney function to a human D-CKD patient in which the improvement in kidney function stabilizes or reduces the patient's uACR, the patient's uACR may stabilize or be reduced from a first time point, e.g., within about one month of administration of either the first or the second injection with the therapeutic composition, to a second time point, e.g., approximately 12 to 24 months after administration of either the first or second injection with the therapeutic composition. The improvement in kidney function may stabilize or reduce the patient's uACR from a first time point, e.g., within about a month of administration of the first injection with the therapeutic composition, to a second time point that may be about 12 to 24 months, e.g., approximately 12 or 18 or 24 months, after administration of the first injection with the therapeutic composition. The improvement in kidney function may stabilize or reduce the patient's uACR from a first time point, e.g., within about a month of administration of the second injection with the therapeutic composition, to a second time point that may be about 12 to 24 months, e.g., approximately 12 or 18 or 24 months, after administration of the second injection with the therapeutic composition. If the improvement in kidney function is a stabilization of the patient's uACR, the patient's uACR may change by less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, or less than about 1% from a first time point, e.g., within about a month of administration of the first or second injection with the therapeutic composition, to a second time point that may be about 12 to 24 months, e.g., approximately 12 or 18 or 24 months, after administration of the first or second injection, respectively, with the therapeutic composition. If the improvement in kidney function is or includes a reduction the patient's uACR, the reduction may be by about 1 to 20%, by about 1 to 19%, by about 1 to 18%, by about 1 to 17%, by about 1 to 16%, by about 1 to 15%, by about 1 to 14%, by about 1 to 13%, by about 1 to 12%, by about 1 to 11%, by about 1 to 10%, by about 1 to 9%, by about 1 to 8%, by about 1 to 7%, by about 1 to 6% or by about 1 to 5%, from a first time point, e.g., within about a month of administration of the first or second injection with the therapeutic composition, to a second time point that may be about 12 to 24 months, e.g., approximately 12 or 18 or 24 months, after administration of the first or second injection, respectively, with the therapeutic composition.

In the methods of improving kidney function to a human D-CKD patient in which the improving kidney function may be a restoration of regulation of erythropoiesis by the kidney, the restoration of regulation of erythropoiesis by the patient's kidney may occur over time from a first time point, e.g., within about one month of administration of either the first or the second injection with the therapeutic composition, to a second time point e.g., approximately 12 to 24 months following administration of the first or second injection, respectively, with the therapeutic composition. If restoration of regulation of erythropoiesis by the patient's kidney is a stabilization of the patient's blood hemoglobin level because the patient's blood hemoglobin level is in the normal range, e.g., between about 13.0 and 17.5 g/dL, then the stabilization of the patient's blood hemoglobin level by the kidney may be a maintenance of the patient's blood hemoglobin level in normal range from a first time point, e.g., within about one month of administration of the first injection with the therapeutic composition, to a second time point e.g., approximately 12 to 24 months (approximately 12 or 18 or 24 months), following the administration of the first injection with the therapeutic composition. If restoration of regulation of erythropoiesis by the patient's kidney is a stabilization of the patient's blood hemoglobin level because the patient's blood hemoglobin level is in the normal range, e.g., between about 13.0 and 17.5 g/dL, then the stabilization of the patient's blood hemoglobin level by the kidney may be maintenance of the patient's blood hemoglobin level in normal range from a first time point, e.g., within about one month of administration of the second injection with the therapeutic composition, to a second time point e.g., approximately 12 to 24 months (approximately 12 or 18 or 24 months), following the administration of the second injection with the therapeutic composition. If restoration of regulation of erythropoiesis by the patient's kidney increases the patient's blood hemoglobin level, because the patient's blood hemoglobin level is below about 13.0 g/dL, and then maintains it in normal range (e.g., between about 13.0 and 17.5 g/dL), then the restoration of regulation of erythropoiesis by the patient's kidney may increase the patient's blood hemoglobin level to at least about 13.0 g/dL and then maintain it in normal range (e.g., between about 13.0 and 17.5 g/dL), from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of erythropoiesis by the patient's kidney increases the patient's blood hemoglobin level, because the patient's blood hemoglobin level is below about 13.0 g/dL, and then maintains it in normal range (e.g., between about 13.0 and 17.5 g/dL), then the restoration of regulation of erythropoiesis by the patient's kidney may increase the patient's blood hemoglobin level to at least about 13.0 g/dL and then maintain it in normal range (e.g., between about 12.0 and 17.5 g/dL), from a first time point, e.g., within about one month of administration of the second injection with the therapeutic composition, to a second time point e.g., approximately 12 to 24 months (approximately 12 or 18 or 24 months) following the administration of the second injection with the therapeutic composition.

In the methods of improving kidney function to a human D-CKD patient in which the improving kidney function may be a restoration of regulation of blood or serum potassium level by the kidney, the restoration of regulation of blood or serum potassium level by the patient's kidney may occur over time from a first time point, e.g., within about one month of administration of either the first or the second injection with the therapeutic composition, to a second time point e.g., approximately 12 to 24 months following administration of the first or second injection, respectively, with the therapeutic composition. If restoration of regulation of blood or serum potassium by the patient's kidney is a stabilization of the patient's blood or serum potassium level because the patient's blood or serum potassium level is in the normal range, e.g., between about 3.5 and 5.0 mmol/L, then the stabilization of the patient's blood or serum potassium level by the kidney may be maintenance of the patient's blood or serum potassium level in normal range from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum potassium by the patient's kidney is a stabilization of the patient's blood or serum potassium level because the patient's blood or serum potassium level is in the normal range, e.g., between about 3.5 and 5.0 mmol/L, then the stabilization of the patient's blood or serum potassium level by the kidney may be maintenance of the patient's blood or serum potassium level in normal range from a first time point, e.g., within about one month of administration of the second injection with the therapeutic composition, to a second time point e.g., approximately 12 to 24 months (approximately 12 or 18 or 24 months) following the administration of the second injection with the therapeutic composition. If restoration of regulation of blood or serum potassium level by the patient's kidney decreases the patient's blood or serum potassium level, because the patient's blood or serum potassium level is greater about 5.0 mmol/L, and then maintains it in normal range (e.g., between about 3.5 and 5.0 mmol/L), then the restoration of regulation of blood or serum potassium level by the patient's kidney may decrease the patient's blood or serum potassium level to at least about 5.0 mmol/L and then maintain it in normal range (e.g., between about 3.5 and 5.0 mmol/L), from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum potassium by the patient's kidney decreases the patient's blood or serum potassium level, because the patient's blood or serum potassium level is greater than about 5.0 mmol/L, and then maintains it in normal range (e.g., between about 3.5 and 5.0 mmol/L), then the restoration of regulation of blood or serum potassium by the patient's kidney may decrease the patient's blood or serum potassium level to at least about 5.0 mmol/L and then maintain it in normal range (e.g., between about 3.5 and 5.0 mmol/L), from a first time point, e.g., within about one month of administration of the second injection with the therapeutic composition, to a second time point e.g., approximately 12 to 24 months (approximately 12 or 18 or 24 months) following the administration of the second injection with the therapeutic composition.

In the methods of improving kidney function to a human D-CKD patient in which the improving kidney function may be a restoration of regulation of blood or serum phosphorus level by the kidney, the restoration of regulation of blood or serum phosphorus level by the patient's kidney may occur over time from a first time point, e.g., within about one month of administration of either the first or the second injection with the therapeutic composition, to a second time point e.g., approximately 12 to 24 months following administration of the first or second injection, respectively, with the therapeutic composition. If restoration of regulation of blood or serum phosphorus by the patient's kidney is a stabilization of the patient's blood or serum phosphorus level because the patient's blood or serum phosphorus level is in the normal range, e.g., between about 3.5 and 5.5 mg/dL, then the stabilization of the patient's blood or serum phosphorus level by the kidney may be maintenance of the patient's blood or serum phosphorus level in normal range from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum phosphorus by the patient's kidney is a stabilization of the patient's blood or serum phosphorus level because the patient's blood or serum phosphorus level is in the normal range, e.g., between about 3.5 and 5.5 mg/dL, then the stabilization of the patient's blood or serum phosphorus level by the kidney may be maintenance of the patient's blood or serum phosphorus level in normal range from a first time point, e.g., within about one month of administration of the second injection with the therapeutic composition, to a second time point e.g., approximately 12 to 24 months (approximately 12 or 18 or 24 months) following the administration of the second injection with the therapeutic composition. If restoration of regulation of blood or serum phosphorus level by the patient's kidney decreases the patient's blood or serum phosphorus level, because the patient's blood or serum phosphorus level is greater about 5.5 mg/dL, and then maintains it in normal range (e.g., between about 3.5 and 5.5 mg/dL), then the restoration of regulation of blood or serum potassium level by the patient's kidney may decrease the patient's blood or serum phosphorus level to at least about 5.5 mg/dl and then maintain it in normal range (e.g., between about 3.5 and 5.5 mg/dL), from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum phosphorus by the patient's kidney decreases the patient's blood or serum phosphorus level, because the patient's blood or serum phosphorus level is greater than about 5.5 mg/dL, and then maintains it in normal range (e.g., between about 3.5 and 5.5 mg/dL), then the restoration of regulation of blood or serum phosphorus by the patient's kidney may decrease the patient's blood or serum phosphorus level to at least about 5.5 mmol/L and then maintain it in normal range (e.g., between about 3.5 and 5.5 mg/dL), from a first time point, e.g., within about one month of administration of the second injection with the therapeutic composition, to a second time point e.g., approximately 12 to 24 months (approximately 12 or 18 or 24 months) following the administration of the second injection with the therapeutic composition.

In the methods of improving kidney function to a human D-CKD patient in which the improving kidney function may be a restoration of regulation of blood or serum calcium level by the kidney, the restoration of regulation of blood or serum calcium level by the patient's kidney may occur over time from a first time point, e.g., within about one month of administration of either the first or the second injection with the therapeutic composition, to a second time point e.g., approximately 12 to 24 months following administration of the first or second injection, respectively, with the therapeutic composition. If restoration of regulation of blood or serum calcium by the patient's kidney is a stabilization of the patient's blood or serum calcium level because the patient's blood or serum calcium level is in the normal range, e.g., between about 8.5 and 10.5 mg/dL, then the stabilization of the patient's blood or serum calcium level by the kidney may be maintenance of the patient's blood or serum calcium level in normal range from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum calcium by the patient's kidney is a stabilization of the patient's blood or serum calcium level because the patient's blood or serum calcium level is in the normal range, e.g., between about 8.5 and 10.5 mg/dL, then the stabilization of the patient's blood or serum calcium level by the kidney may be maintenance of the patient's blood or serum calcium level in normal range from a first time point, e.g., within about one month of administration of the second injection with the therapeutic composition, to a second time point e.g., approximately 12 to 24 months (approximately 12 or 18 or 24 months) following the administration of the second injection with the therapeutic composition. If restoration of regulation of blood or serum calcium level by the patient's kidney increases the patient's blood or serum calcium level, because the patient's blood or serum calcium level is less about 8.5 mg/dL, and then maintains it in normal range (e.g., between about 8.5 and 10.5 mg/dL), then the restoration of regulation of blood or serum calcium level by the patient's kidney may increase the patient's blood or serum calcium level to at least about 8.5 mg/dL and then maintain it in normal range (e.g., between about 8.5 and 10.5 mg/dL), from a first time point, e.g., within about one month of administration of the first injection with the therapeutic composition, to a second time point e.g., approximately 12 to 24 months (approximately 12 or 18 or 24 months) following the administration of the first injection with the therapeutic composition. If restoration of regulation of blood or serum calcium by the patient's kidney increases the patient's blood or serum calcium level, because the patient's blood or serum calcium level is less than about 8.5 mg/dL, and then maintains it in normal range (e.g., between about 8.5 and 10.5 mg/dL), then the restoration of regulation of blood or serum calcium by the patient's kidney may increase the patient's blood or serum calcium level to at least about 8.5 mmol/L and then maintain it in normal range (e.g., between about 8.5 and 10.5 mg/dL), from a first time point, e.g., within one month of administration of the second injection with the therapeutic composition, to a second time point e.g., approximately 12 to 24 months (approximately 12 or 18 or 24 months) following the administration of the second injection with the therapeutic composition.

In the methods of improving kidney function of a human D-CKD patient in which the improving kidney function increases blood filtration by the patient's kidney, the increase in blood filtration by the patient's kidney may be from a first time point, e.g., within about one month of administration of either the first or the second injection with the therapeutic composition, to a second time point e.g., approximately 12 to 24 months following administration of the first or second injection, respectively, with the therapeutic composition. The patient's improvement in kidney function, if it is an increase in blood filtration by the patient's kidney, may be from a first time point, e.g., within about a month of the first injection with the therapeutic composition, to a second time point that may be about 12 to 24 months, e.g., approximately 12 or 18 or 24 months, after administration of the first injection with the therapeutic composition. The patient's improvement in kidney function may be an increase in blood filtration by the patient's kidney from a first time point, e.g., within about a month of the second injection with the therapeutic composition, to a second time point that may be about 12 to 24 months, e.g., approximately 12 or 18 or 24 months, after administration of the second injection with the therapeutic composition. The patient's improvement in kidney function may be an increase in kidney function of between about 1% and 15%, between about 1% and 10%, between about 1% and 5%, between about 5% and 15% or between about 5% and 10% from a first time point that may be within about a month of administration of the first or second injection with the therapeutic composition to a second time point that may be about 12 to 24 months, e.g., approximately 12 or 18 or 24 months, after administration of the first or second injection, respectively, with the therapeutic composition. The patient's improvement in kidney function, if the improvement is determined as a stabilization in eGFR, may be a reduction in rate of decline in eGFR (e.g., by at least about 25%, 30%, 35%, 40%, 45%, 50%, 55%, 60%, 65%, 70%, 75%, 80%, 85%, 90%, 95% or 100%) from a first time point that may be within about a month of administration of the first or second injection with the therapeutic composition to a second time point that may be about 12 to 24 months, e.g., approximately 12 or 18 or 24 months, after administration of the first or second injection, respectively, with the therapeutic composition. The patient's improvement in kidney function may be an increase in blood filtration by the patient's kidney that continues over time, e.g., in which blood filtration by the patient's kidney increases from the first time point to about the midpoint between the first and the second time point, and again increases from between about the midpoint between the first and the second time point to the second time point. The increases in blood filtration by the patient's kidney need not be identical. For example, the improvement in kidney function may be an increase such filtration by the patient's kidney increases from the first time point to about 6 months from the first time point, and again increases from about 6 months from the first time point to the second time point if the first time point is within about a month from the administration of the first or second injection of the therapeutic composition and the second time point is about 12 months after administration of the first or second, respectively, injection of the therapeutic composition. Again the increases need not be the same, e.g., identical.

Because administration of the first and second doses of therapeutic composition, e.g., including SRCs, can improve kidney function, the methods improving kidney function may further prevent renal replacement therapy, e.g., dialysis or kidney transplant, of the patient. The prevention of renal replacement therapy of the patient may be a prevention of the need for dialysis of the patient. The prevention of the need for dialysis of the patient may be for at least 5 years, at least 6 years, at least 7 years, at least 8 years, at least 9 years, at least 10 years, at least 11 years, at least 12 years, at least 13 years, at least 14 years, at least 15 years, at least 16 years, at least 17 years, at least 18 years, at least 19 years, or at least 20 years from the administration of the first or second injection of the therapeutic composition. Dialysis of the patient may be hemodialysis or peritoneal dialysis (continuous ambulatory peritoneal dialysis, continuous cyclic peritoneal dialysis or intermittent peritoneal dialysis) of the patient. The prevention of renal replacement therapy of the patient may be a prevention of the need for kidney transplant of the patient. The prevention of the need for kidney transplant of the patient may be for at least 5 years, at least 6 years, at least 7 years, at least 8 years, at least 9 years, at least 10 years, at least 11 years, at least 12 years, at least 13 years, at least 14 years, at least 15 years, at least 16 years, at least 17 years, at least 18 years, at least 19 years, or at least 20 years from the administration of the first or second injection of the therapeutic composition.

The improvement of kidney function, e.g., having administered the first and second injections of a therapeutic composition, e.g., including SRCs, to D-CKD patients as disclosed herein is believed to be the first demonstration of any therapeutic, or therapeutic regimen, capable of improving a D-CKD patient's kidney function. Of particular note, not only did D-CKD patients receiving two injections of the therapeutic, SRC-containing, composition exhibit improved kidney function, they exhibited improved, or improving, kidney function over an extended period of time, e.g., at least one year. The effects, and sustained effects, from administration of the two injections with the SRC-containing therapeutic composition offer the ability to prevent renal replacement therapy of D-CKD patients, and may reduce the need of these patients for (or dependency on) treatments to ameliorate co-morbidities of CKD, e.g., EPO to ameliorate anemia. Beyond these clinical findings, because D-CKD patients who received the two injections with the SRC-containing therapeutic composition showed kidney function improvement, SRCs may be capable of triggering or mediating nephrogenic-like activity in the diseased kidneys of the adult D-CKD patients. This implication is also significant as the regeneration of nephrons, or neo-nephrogenesis, has been believed to substantially conclude in humans around the 36th week of gestation.

These improvement in kidney function, e.g., having administered the first and second injections of the SRC-containing therapeutic composition as disclosed herein, were further observed in patients in late stage IV D-CKD, e.g., patient's having eGFRs of less than about 20 mL/min/1.73 m². D-CKD patients nearing Stage V D-CKD face the challenge of few available therapies that slow the rapid trajectory to end stage kidney disease. Most management algorithms at this stage of D-CKD focus on D-CKD comorbidities, such as ardent hypertension control. Further, at this stage of DKD, patients and families recognize the time to dialysis is impending. Nonetheless, administration of first and second injections of the SRC therapeutic, as disclosed herein, resulted in improvement in kidney filtration, e.g., reduction in rate of decline in eGFR, and delayed time to renal replacement therapy, e.g., dialysis. For instance, the kidney function of D-CKD patients having an eGFR of less than approximately 20 ml/min/1.73 m², having been administered first and second injections of the SRC-containing therapeutic, experienced a reduction in rate of decline in eGFR (of up at least 25%, 30%, 35%, 40%, 45%, 50%, 55%, 60%, 65%, 70%, 75%, 80% 85%, 90%, 95%), and prevention of renal replacement therapy, e.g., delay in dialysis or kidney transplant (by at least about 4 months, 6 months, 1, year, 1.5 years, 2 years, 2.5 years, 3 years, 4 years, etc.).

SRCs or cells of a SRC population, administered in a therapeutic composition, being capable of improving a CKD patient's kidney function, thus, include bioactive renal cells, or cells having biological, e.g., pharmacological or therapeutic, activity. Biological, e.g., pharmacological or therapeutic, activities of the SRC's bioactive cells may include any one or more of: enhancing renal function, improving renal homeostasis, promoting renal tissue healing, reducing renal tissue fibrosis, reducing renal tissue inflammation, promoting renal regeneration or promoting renal tissue repair.

SRCs, or cells of the SRC population, administered in a therapeutic composition that improves a patient's kidney function in the methods provided herein, may be prepared from kidney tissue of a patient, a kidney biopsy of a patient, or an in vitro culture of cells established from kidney tissue or a kidney biopsy of a patient, (which, collectively, may be referred to as a “starting renal cell population”). A starting renal cell population, if an in vitro culture of cells established from a kidney tissue of a patient or a kidney biopsy of a patient, may be a renal cell preparation including dissociated cells of a kidney tissue or kidney biopsy (e.g., cells dissociated from the kidney tissue or kidney biopsy via mincing and/or enzyme digestion), that may or may not have been treated to remove red blood cells and debris.

SRCs, or cells of the SRC population, administered in a therapeutic composition that improves a patient's kidney function in the methods provided herein, may be prepared from the starting renal cell population, (e.g., a kidney tissue of a patient, a kidney biopsy of a patient, or an in vitro culture of cells established from kidney tissue or kidney biopsy of a patient), via a method that includes a separation step. The separation step may be a step that separates cells of the starting renal cell population that have passaged no more than one, two, or three times, on the basis of their buoyant density. If the separation step is one that separates cells on the basis of their buoyant density, the separation step may utilize a single or multi-step continuous or discontinuous density gradient using a density gradient media such as glycerol, glucose OptiPrep™, Percoll®, or Ficoll®-Paque. The use of such a density gradient media in this manner may result in cells of the starting renal cell population (or starting renal cell population having been passaged at most one, two or three times) separating into one or more distinguishable fractions, e.g., which may be a cell pellet, from which SRCs or cells of the SRC population may be distinctly identified and isolated. The distinguishable fraction(s) may be those in which the buoyant density of cells in the fraction(s) is greater than about 1.045 g/mL, or greater than 1.045 g/mL, or greater than or equal to 1.045 g/mL. The distinguishable fraction(s) may be those in which the buoyant density of cells in the fraction(s) is greater than about 1.04 g/mL, or greater than 1.04 g/mL, or greater to or equal than 1.04 g/mL, or greater than about 1.0419 g/mL, or greater than 1.0419 g/mL, or greater to or equal than 1.0419 g/mL. The distinguishable fraction(s) may be those in which the buoyant density is between about 1.045 g/mL and about 1.091 g/mL, or between about 1.045 g/mL and about 1.052 g/mL. Alternatively, the separation step may be one that separates cells of the starting renal cell population (or cells of the starting renal cell population that have been passaged no more than one, two or three times), on the basis of whether they express particular markers on their surface. If the separation step separates cells on the basis of their expression of particular cell surface markers, the separation step may be one that utilizes flow cytometry. The flow cytometry may sort cells from the starting renal cell population (or starting renal cell population having been passaged at most one, two or three times) based on their expression of particular surface markers, such as nephrin, that are characteristic of renal epithelial cells, renal tubular cells, renal tubular epithelial cells, or renal proximal tubular cells, to isolate the SRCs.

SRCs, or cells of the SRC population, administered in a therapeutic composition that improves a patient's kidney function in the methods provided herein, may further be hypoxia resistant. The SRCs, or cells of the SRC population, may further be hypoxia resistant due to the cells of the starting renal cell population (or cells of the starting renal cell population that have been passaged no more than one, two or three times) having been subject to hypoxic conditions, e.g., prior to the separation step. If cells of the starting renal cell population (or cells of the starting renal cell population having been passaged no more than one, two or three times) have been subject to hypoxic conditions, the cells of the starting renal cell population (or cells of the starting renal cell population having been passaged no more than one, two or three times) may have been cultured at oxygen levels of less than about 20%, or less than about 15%, or less than about 10%, or less than about 9%, or less than about 8%, or less than about 7%, or less than about 6%, or less than about 5% oxygen, or less than about 4% oxygen, or less than about 3% oxygen or less than about 2% oxygen. The cells may have been cultured at the oxygen levels of less than about 20%, 15%, 10%, 9%, 8%, 7%, 6%, 5%, 4%, 3%, or 2% for at least 6 hours, at least 8 hours, at least 10 hours, at least 12 hours, at least 14 hours, at least 16 hours, at least 20 hours, at least 24 hours, at least 30 hours, at least 36 hours, at least 42 hours, at least 48 hours between about 6 to 48 hours, between about 6 to 24 hours, between about 6 to 12 hours between about 12 hours and 48 hours, between about 12 hours and 24 hours, between about 12 hours and 18 hours, between about 18 hours and 48 hours or between about 18 hours and 24 hours.

In general, SRCs (or cells of the SRC population) may be prepared from a starting renal cell population, e.g., an in vitro culture of cells established from a kidney tissue of a patient or a kidney biopsy of a patient. The starting renal cell population, e.g., in vitro culture of cells established from the kidney tissue or kidney biopsy of the patient, may be expanded by passaging at most one, or at most two or at most three times. Alternatively, if desired, cells of the in vitro culture of cells established from the kidney tissue or kidney biopsy may be cryopreserved and then expanded by the passaging at most one, or at most two or at most three times. Once the cells have been expanded, they may be cryopreserved. The expanded cells, whether or not cryopreserved, may be subject to a separation step or may be subject to hypoxic culture conditions followed by a separation step. The SRCs are isolatable by having performed the separation step. Once the SRCs have been isolated, they may be frozen and/or analyzed prior to use in a therapeutic composition.

SRCs, or cells of the SRC population, administered in a therapeutic composition that improves a patient's kidney function in the methods provided herein, may be described as including bioactive renal cells or hypoxia-resistant bioactive renal cells prepared from a starting renal cell population. SRCs, or cells of a SRC population, administered in a therapeutic composition that improves a patient's kidney function in the methods provided herein, may be described as including bioactive renal cells or hypoxia-resistant bioactive renal cells prepared from a starting renal cell population (or cells of the starting renal cell population having been passaged no more than one, two or three times) following a separation step.

SRCs, or cells of a SRC population may further be described, e.g., in addition to being described as bioactive renal cells or hypoxia-resistant bioactive renal cells prepared from a starting renal cell population (or cells of the starting renal cell population having been passaged no more than one, two or three times that may or may not have been subjected to a separation step), according to any of a number of their other characteristics. For instance, SRCs or cells of the SRC population may be further described, e.g., in addition to being described as bioactive renal cells or hypoxia-resistant bioactive renal cells prepared from a starting renal cell population (or cells of the starting renal cell population having been passaged no more than one, two or three times that may or may not have been subjected to a separation step), according to characteristic cell types included therein. For example, the SRCs or cells of the SRC population may be further described as including renal epithelial cells, renal tubular cells, renal tubular epithelial cells, or renal proximal tubular cells. The SRCs or cells of the SRC population may further be described as including these renal cell types, (e.g., renal epithelial cells, renal tubular cells, renal tubular epithelial cells or renal proximal cells) at higher percentages than did the starting renal cell population. SRCs, or cells of the SRC population, may include these renal cell types (e.g., renal epithelial cells, renal tubular cells, renal tubular epithelial cells or renal proximal cells) at higher percentages than did the starting renal cell population due to the subjecting of the starting renal cell population (or cells of the starting renal cell population having been passaged no more than one, two or three times) to a separation step. The SRCs or cells of the SRC population, may be described as including renal epithelial cells, renal tubular cells, renal tubular epithelial cells and/or renal proximal cells at higher percentages than did the starting renal cell population and as additionally including other renal cell types, such as glomerular cells, podocytes, collecting duct cells and/or vascular cells.

As another example, SRCs or cells of the SRC population may further be described, e.g., in addition to being described as bioactive renal cells or hypoxia-resistant bioactive renal cells prepared from a starting renal cell population (or cells of the starting renal cell population having been passaged no more than one, two or three times that may or may not have been subjected to a separation step), according to their including tubular, glomerular and/or peritubular interstitial renal cell types.

Alternatively, SRCs or cells of the SRC population may be further described, e.g., in addition to being described as bioactive renal cells or hypoxia-resistant bioactive renal cells prepared from a starting renal cell population (or cells of the starting renal cell population having been passaged no more than one, two or three times) according to a characteristic for which the SRCs may have been selected at a separation step, if performed, e.g. buoyant density. If the SRCs, or cells of the SRC population, are prepared from the starting renal cell population (or cells of the starting renal cell population having been passaged no more than one, two or three times) by a method employing a buoyant density separation step, then the SRCs may further be characterized as renal cells prepared from a starting kidney cell population (or cells of the starting renal cell population having been passaged no more than one, two or three times) having a buoyant density of greater than about 1.045 g/mL, greater than 1.045 g/mL, greater than or equal to about 1.045 g/mL, greater than about 1.04 g/mL, greater than 1.04 g/mL, or greater than or equal to about 1.04 g/mL, greater than about 1.0419 g/mL, greater than 1.0419 g/mL, greater than or equal to about 1.0419 g/mL between about 1.045 g/mL and about 1.091 g/mL or between about 1.045 g/mL and about 1.052 g/mL.

In a different example, SRCs, or cells of a SRC population may further be described, e.g., in addition to being described as including bioactive renal cells or hypoxia-resistant bioactive renal cells prepared from a starting renal cell population (or cells of the starting renal cell population having been passaged no more than one, two or three times, and that may or may not have been subjected to a separation step), according to their surface marker expression characteristics and/or secretion of certain products. For example, the SRCs or cells of the SRC population may further be described as including cells that express gamma-glutamyl transpeptidase (GGT)-1 and/or cells that express a cytokeratin (CK). If the SRCs, or cells of the SRC population, are further described as including cells that express GGT-1, then the SRCs or cells of the SRC population may further be described as cells or a cell population in which between about 4% and 82%, between about 4.5% and 80%, between about 4.5% and 75%, between about 4.5% and 70%, at least about 4.5%, at least about 10%, at least about 15% or at least about 20% of the cells express GGT-1. If the SRCs, or cells of the SRC population are further described as including cells that express a CK, e.g., CK18, then the SRCs or cells of the SRC population may further be described cells or a cell population in which between about 80% and 100%, between about 80% and 97%, at least 80%, at least 82%, at least 84%, at least 86%, at least 88%, at least 90%, at least 92% or at least 94% of the cells express the CK, e.g., CK18. If the SRCs, or cells of the SRC population, are further described as including cells that express GGT-1 and cells that express CK18, then the SRCs or cells of the SRC population may further be described as cells or a cell population in which between about 4% and 82% of the cells express GGT-1 and between about 80% and 100% of the cells express CK18, or at least about 4.5% of cells express GGT-1 and at least about 80% of cell express CK18, or at least about 10% of cells express GGT-1 and at least about 82% of cell express CK18, or at least about 18% of cells express GGT-1 and at least about 82% of cell express CK18. The SRCs or cells of the SRC population, may further be described by not only including cells that express GGT-1 and cells that express a CK, e.g., CK18, but also by including cells that secrete vascular endothelial growth factor (VEGF) and/or kidney injury molecule-1 (KIM-1). In such instances, the SRCs or cells of the SRC population may further be described as cells or a cell population in which: cells secrete VEGF, cells secrete KIM-1, between about 4% and 82% of the cells express GGT-1 and between about 80% and 100% of the cells express CK18; or cells secrete VEGF, cells secrete KIM-1, at least about 4.5% of the cells express GGT-1 and at least about 80% of the cells express CK18; or cells secrete VEGF, cells secrete KIM-1, at least about 10% of the cells express GGT-1 and at least about 82% of the cells express CK18; or cells that VEGF, cells secrete KIM-1, at least about 18% of the cells express GGT-1 and at least about 82% of the cells express CK18.

Alternatively, SRCs or cells of a SRC population may further be described, e.g., in addition to being described as bioactive renal cells or hypoxia-resistant bioactive renal cells prepared from a starting renal cell population (or cells of the starting renal cell population having been passaged no more than one, two or three times, and that may or may not have been subjected to a separation step), according to their nephrogenic marker expression characteristics. For instance, the SRCs or cells of the SRC population may further be described by their inclusion of renal cells that express one or more of nephrogenic markers SIX Homeobox 2 (SIX2), odd-skipped-related 1 (OSR1), LIM homeobox 1 (LHX1), rearranged during transfection (RET) or fibroblast growth factor 8 (FGF8). The SRCs or cells of a SRC population, if further described by their inclusion of renal cells that express one or more nephrogenic markers, may further be described by their inclusion of renal cells that express LHX1, or that express SIX2 or that express both LHX1 and SIX2. If the SRCs, or cells of the SRC population are further described as including cells that express a LHX1, the SRCs or cells of the SRC population may further be described as cells or a cell population in which at least about 15%, at least about 20%, at least about 25%, at least about 30%, at least about 35%, at least about 40%, at least about 45%, at least about 50%, at least about 55%, at least about 60%, at least about 65%, at least about 70%, between about 15% and about 80%, between about 20% and about 80%, between about 15% and about 75%, between about 15% and about 70%, or between about 20% and about 80% of the cells express LHX1. If the SRCs or cells of a SRC population are further described as including cells that express SIX2, the SRCs or cells of the SRC population may further be described as cells or a cell population in which between about 0.02% to about 15.0%, or between about 0.02% to about 10.0%, or between about 0.02% to about 9.0%, or between about 0.02% and about 8.0%, or between about 0.02% and about 7.0%, or between about 0.02% and about 6.0%, or between about 1.0% to about 15.0%, or between about 1.0% to about 10.0%, or between about 1.0% to about 9.0%, or between about 1.0% and about 8.0%, or between about 1.0% and about 7.0% or between about 1.0% and about 6.0% of the cells express SIX2. If the SRCs or cells of the SRC population are further described as including cells that express RET, the SRCs or cells of the SRC population may further be described as cells or a cell population in which between about 45% and about 95%, or between about 45% and about 94%, or between about 45% and about 93%, or between about 45% and about 92%, or between about 45% and about 91%, or between about 45% and about 90%, or between about 45% and about 89%, or between about 45% and about 88% of the cells express RET. If the SRCs or cells of the SRC population are described as including cells that express OSR1, the SRCs or cells of the SRC population may further be described as cells or a cell population in which between about 30% and about 86%, or between about 30% and about 84%, or between about 30% and about 82%, or between about 30% and about 80%, or between about 40% and about 90%, or between about 40% and about 85%, or between about 45% and about 90%, or between about 45% and about 85%, or between about 50% and about 90%, or between about 50% and about 85% of the cells express OSR1. If the SRCs or cells of the SRC population are described as including cells that express FGF8, then the SRCs or cells of the SRC population may further be described as cells or a cell population in which greater than 0% and up to at most about 60%, or greater than 0% and up to at most about 59%, or at most about 58%, or greater than 0% and up to at most about 57%, or greater than 0% and up to at most about 56% or greater than 0% and up to at most about 55%, or between about 1% and about 64%, or between about 1% and about 54%, or between about 1% and about 44%, or between about 1% and about 34%, or between 1% and about 24% of the cells express FGF8.

SRCs, or cells of a SRC population may further be described, e.g., in addition to being described as bioactive renal cells or hypoxia-resistant bioactive renal cells prepared from a starting renal cell population (or cells of the starting renal cell population having been passaged no more than one, two or three times, and that may or may not have been subjected to a separation step), according to their nephrogenic marker and other surface marker expression characteristics. For instance, the SRCs or cells of the SRC population may further be described as including renal cells that express nephrin, podocin and LHX1. If the SRCs or cells of the SRC population are further described by inclusion of cells that express nephrin, podocin and LHX1, the SRCs or cells of the SRC population may further be described as cells or a cell population in which (i) at least about 70%, at least about 72%, at least about 75%, at least about 77%, at least about 80%, at least about 82%, at least about 85%, at least about 87%, at least about 90%, at least about 92%, at least about 95%, or at least about 97% of the cells express nephrin; and/or (ii) at least about 80%, at least about 82%, at least about 84%, at least about 86%, at least about 88%, at least about 90%, at least about 92%, at least about 94%, at least about 96% or at least about 98% of the cells express podocin; and/or (iii) at least about 15%, at least about 20%, at least about 25%, at least about 30%, at least about 35%, at least about 40%, at least about 45%, at least about 50%, at least about 55%, at least about 60%, at least about 65%, at least about 70%, between about 15% and about 80%, between about 20% and about 80%, between about 15% and about 75%, between about 15% and about 70%, or between about 20% and about 80% of the cells express LHX1.

The SRCs, or cells of the SRC population, may be formulated in the therapeutic composition in a hydrogel or a liquid composition form. The hydrogel or liquid composition form may, or may not, include hyaluronic acid.

If the therapeutic composition is in a hydrogel composition form, the SRCs or cells of the SRC population may be combined with a temperature-sensitive cell-stabilizing biomaterial. The temperature-sensitive cell-stabilizing biomaterial may be a biomaterial in a gel state at certain temperatures and a liquid state at others. For example, if the biomaterial is temperature-sensitive, the biomaterial may be in a gel state at about 8° C. or below, a substantially liquid state at about ambient temperature or above, and a solid-to-liquid transitional state between about 8° C. and about ambient temperature; or a gel state at about 4° C. or below, a liquid state at about 37° C. or above, and a solid-to-liquid transitional state between about 8° C. and about 18° C.; or a gel state at about 2° C. or below, a liquid state at about 37° C. or above, and a solid-to-liquid transitional state between about 8° C. and about 18° C.; or a gel state at about 2° C. or below and a liquid state at about 37° C. or above; or a gel state at about 4° C. or below and a liquid state at about 34° C. or above; or a gel state at about 6° C. or below and a liquid state at about 32° C. or above.

Furthermore, if the therapeutic composition is in a hydrogel composition form, and the SRCs or cells of the SRC population are combined with a temperature-sensitive cell-stabilizing biomaterial, the biomaterial may include, or be made up of, one or more naturally sourced or recombinant proteins or peptides. The naturally sourced or recombinant proteins or peptides may be extracellular matrix proteins of recombinant origin, or extracellular matrix sourced from kidney or another tissue or organ, or gelatin. If the temperature-sensitive cell-stabilizing biomaterial is, or includes, gelatin, the gelatin may be derived from a Type I, alpha I collagen such as porcine Type I, alpha I collagen or recombinant human Type I, alpha I collagen. If the temperature-sensitive cell-stabilizing biomaterial is, or includes, gelatin, the gelatin may present in the therapeutic composition at about 0.5% to about 1% weight per volume (w/v), or about 0.8% to about 0.9% (w/v), or about 0.75% (w/v) or about 0.88% (w/v). SRCs or cells of the SRC population may be formulated in the biomaterial, e.g., gelatin, such that the number of SRC per mL biomaterial is about 20×10⁶ cells per mL, about 40×10⁶ cells per mL, about 60×10⁶ cells per mL, about 100×10⁶ cells per mL, about 120×10⁶ cells per mL, about 140×10⁶ cells per mL, about 160×10⁶ cells per mL, about 180×10⁶ cells per mL, or about 200×10⁶ cells per mL.

SRCs or cells of the SRC population, when combined with the biomaterial, may be dispersed throughout the biomaterial, e.g., gelatin. SRCs or cells of the SRC population, when combined with the biomaterial, may be substantially uniformly distributed throughout the biomaterial. SRCs or cells of the SRC population, when combined with the biomaterial, may be dispersed throughout the biomaterial, e.g., gelatin, such that they do not settle or aggregate in the biomaterial.

Alternatively, the therapeutic composition may be a liquid composition. If the therapeutic composition is a liquid composition, the SRCs or cells of the SRC population may be combined with any suitable liquid, e.g. appropriate cell storage or culture medium, a saline, or combinations thereof, for immediate use or for cryopreservation up until the timing of its use. If the therapeutic composition is a liquid composition, the SRCs or cells of the SRC population may be suspended in a pharmaceutically acceptable carrier or excipient, such as saline, buffered saline, dextrose, water, polyethyleneglycol, and/or any combinations thereof.

In the methods of improving kidney function to the patient having D-CKD, the therapeutic compositions, e.g., compositions including SRCs, may be administered to the D-CKD patient by injection, e.g., by injection into the renal cortex of at least one kidney of the patient. The injection may be via direct laparotomy, via direct laparoscopy, transabdominally, or percutaneously. The therapeutic composition, composition including SRCs, may be administered to the D-CKD patient by percutaneous injection into the renal cortex of a kidney, or may be administered by inserting a guiding cannula percutaneously to puncture the kidney capsule and then injecting the enriched heterogeneous renal cell population into the kidney. The administration of the therapeutic composition, including SRCs, may be by injection into the renal cortex of one or both kidneys of the patient. The administration of the therapeutic composition, including SRCs, may be by two injections wherein the first injection is into the renal cortex of one kidney and a second injection is into the renal cortex of the other kidney of the patient.

The administration of the therapeutic composition, e.g., composition including SRCs, if administered by injection, may inject a therapeutically effective dose of SRCs into the renal cortex of the at least one kidney of the patient. The therapeutically effective dose of SRCs may be determined based on the estimated weight of the patient's kidney, e.g., in grams. The therapeutically effective dose may include about 1.0-9.0×10⁶ SRCs per gram estimated kidney weight (SRCs/g KWest) of the patient. The therapeutically effective dose may include about 1.0×10⁶, about 2.0×10⁶, about 3.0×10⁶, about 4.0×10⁶, about 5.0×10⁶, about 6.0×10⁶, about 7.0×10⁶, about 8.0×10⁶, about 9.0×10⁶, about 2.0-7.0×10⁶, between about 4.0-7.0×10⁶, or between about 5.0×10⁶-7.0×10⁶ SRCs/g KWest of the patient.

The administration of the therapeutic composition that includes the SRCs, in the methods of improving kidney function to the D-CKD patient, may be by first and second injections. The first and second injections may be administered between approximately 3 and 12 months apart. The first and second injections may be administered approximately 3 months apart, approximately 4 months apart, approximately 5 months apart, approximately 6 months apart, approximately 7 months apart, approximately 8 months apart, approximately 9 months apart, approximately 10 months, approximately 11 months apart or approximately 12 months apart. The first and second injections may be administered between approximately 3 and 6 months apart, between approximately 6 and 9 months apart, between approximately 9 and 12 months apart, between approximately 3 and 9 months apart, between approximately 6 and 9 months apart or between approximately 6 and 12 months apart.

The administration of the therapeutic composition that includes the SRCs, in the methods of improving kidney function to the D-CKD patient, may be by first and second injections where the second injection, if administered, is not necessarily administered at a pre-set, e.g., approximately 3 to 12 months, time after the first injection. Rather, the second injection, if administered, may be administered to the D-CKD patient if a triggering event is detected, so long as the triggering event is detected during a time interval following the first injection.

If a second injection is administered to the D-CKD patient following a triggering event detected during a time interval following the first injection, the time interval may be between approximately 2 months and 80 months, or between approximately 2 months and 74 months, or between approximately 2 months and 70 months, or between approximately 2 months and 64 months, or between approximately 2 months and 60 months, or between approximately 2 months and 56 months, or between approximately 2 months and 52 months, or between approximately 2 months and 48 months, or between approximately 2 months and 44 months, or between approximately 2 months and 40 months, or between approximately 2 months and 36 months, or between approximately 2 months and 32 months, or between approximately 2 months and 28 months, or between approximately 2 months and 24 months, or between approximately 2 months and 20 months, or between approximately 2 months and 18 months, or between approximately 3 months and 80 months, or between approximately 3 months and 74 months, or between approximately 3 months and 70 months, or between approximately 3 months and 64 months, or between approximately 3 months and 60 months, or between approximately 3 months and 56 months, or between approximately 3 months and 52 months, or between approximately 3 months and 48 months, or between approximately 3 months and 44 months, or between approximately 3 months and 40 months, or between approximately 3 months and 36 months, or between approximately 3 months and 32 months, or between approximately 3 months and 28 months, or between approximately 3 months and 24 months, or between approximately 3 months and 20 months, or between approximately 3 months and 18 months after administration of the first injection in the D-CKD patient.

If the second injection is administered to the D-CKD patient following a triggering event detected during a time interval, e.g., between 2 and 36 months, following the first injection, then the second injection may be administered to the D-CKD within about 30 days of having detected the triggering event. If triggering event is detected during a time interval, e.g., between 2 and 36 months, following the first injection, then the second injection may be administered to the D-CKD within about 14 days, 16 days, 18 days, 20 days, 22 days, 24 days, 26 days, 28 days, 30 days, 32 days, 34 days, 36 days, 38 days, 40 days, 42 days, 44 days, or 46 days of having detected the triggering event. It may be administered about 2 to 8 weeks, 2 to 7 weeks, 2 to 6 weeks, 2 to 5 weeks, 2 to 4 weeks, 4 to 8 weeks, 4 to 6 weeks, 3 to 6 weeks, or 4 to 5 weeks after having detected the triggering event.

The triggering event, which if detected during the time interval (e.g., between 2 and 36 months) following the first injection triggers the administration of the second injection, may be a detected reduction or insignificant change, in the patient's eGFR from baseline, e.g., reduction or insignificant change relative to the patient's baseline eGFR. The triggering event, which triggers the second injection with the therapeutic composition, may be a detected increase or insignificant change in the patient's uACR from baseline, e.g., increase or insignificant change relative the patient's baseline uACR. An insignificant change in a patient's baseline eGFR or uACR may be an about 0.0% change from the patient's baseline eGFR or uACR or it may be a slight increase or decrease (e.g., within about 2.0%) change from the patient's baseline eGFR or uACR.

The baseline eGFR or uACR of the patient may be the patient's eGFR or uACR at any time between approximately 90 days prior to the first injection and the day of the first injection. The baseline eGFR or uACR of the patient may be the patient's eGFR or uACR at any time between approximately 60 days prior to administration of the first injection and the day of the first injection. The baseline eGFR or uACR of the patient may be the patient's eGFR or uACR at any time between approximately 45, 30, or 15 days prior to administration of the first injection and the day of the first injection. The baseline eGFR or uACR of the patient may be the eGFR or uACR of the patient at approximately 90 days, approximately 85 days, approximately 80 days, approximately 75 days, approximately 70 days, approximately 65 days, approximately 60 days, approximately 55 days, approximately 50 days, approximately 45 days, approximately 40 days, approximately 35 days, approximately 30 days, approximately 25 days, approximately 20 days approximately 15 days, approximately 10 days, approximately 5 days prior to administration of the first injection, or on the day of the first injection. The baseline eGFR or uACR of the patient may be an average of two or three or four eGFR or uACR measurements of the patient between approximately 90, 60, 45, 30 or 15 days prior to administration of the first injection and the day of the first injection.

The triggering event, detection of which during the time interval after the first injection that may trigger administration of the second injection, may be an insignificant change in the patient's eGFR relative to baseline or may be a reduction in the patient's eGFR of about 5% relative to baseline. The reduction, or insignificant change, in the patient's eGFR, relative to baseline, may be a reduction of about 0.0%, 2.5%, 5.0%, 7.5%, 10.0%, 12.5%, 15.0%, 17.5%, 20.0%, 22.5%, 25.0%, 27.5 or 30.0%. The triggering event may be a reduction in the patient's eGFR of about 0-5% or 5% relative to baseline, if the patient's baseline eGFR is at most about 25 ml/min/1.73 m². The triggering event may be a reduction in the patient's eGFR of about 0-10% or 10% relative to baseline, if the patient's baseline eGFR is between approximately 25 to 35 ml/min/1.73 m². The triggering event may be a reduction in the patient's eGFR of about 0-15% or 15% relative to baseline, if the patient's baseline eGFR is between approximately 30 to 40 ml/min/1.73 m². The triggering event may be a reduction in the patient's eGFR of about 0-20% or 20% relative to baseline, if the patient's baseline eGFR is between approximately 35 to 45 ml/min/1.73 m². The triggering event may be a reduction in the patient's eGFR of about 0-25% or 25% relative to baseline, if the patient's baseline eGFR is at least about 40 ml/min/1.73 m². The triggering event may be a reduction in the patient's eGFR of about 0-30% or 30% relative to baseline, if the patient's baseline eGFR is at least about 45 ml/min/1.73 m². The triggering event, detection of which during the time interval after the first injection that may trigger administration of the second injection, may be detected by having performed a step of determining the patient eGFR at a time point following administration of the first injection. The time point(s) at which the patient's eGFR is determined may be at least one week, two weeks, six weeks, eight weeks, ten weeks, twelve weeks, fourteen weeks, sixteen weeks, eighteen weeks, twenty weeks, twenty two weeks twenty four weeks, seven months, eight months, ten months, twelve months, fourteen months, sixteen months, eighteen months, twenty months, twenty two months or twenty four months following the first injection.

The triggering event, detection of which during the time interval after the first injection may trigger administration of the second injection, may be an insignificant change in the patient's uACR relative to baseline or may be an increase of about 20% relative to baseline. The triggering event, detection of which during the time interval after the first injection that may trigger administration of the second injection, may be a detection of an increase in the patient's uACR of at least about 2.5%, 5.0%, 7.5%, 10.0%, 12.5%, 15%, 17.5%, 20.0%, 22.5%, 25.0%, 27.5%, 30.0%, 32.5%, 35.0%, 37.5%, 40.0%, 42.5%, 45%, 47.5% or 50.0% relative to baseline. The increase in the patient's uACR, relative to baseline, may be by at least about 20% and at least about 30 mg/g. The increase in the patient's uACR, relative to baseline, in addition to being an increase of at least about 30 mg/g, may be an increase of about 2.5%, 5.0%, 7.5%, 10.0%, 12.5%, 15%, 17.5%, 20.0%. The triggering event, detection of which during the time interval after the first injection that may trigger administration of the second injection, may be detected by having performed a step of determining the patient uACR at least one time point following administration of the first injection. The time point(s) at which the patient's uACR is determined may be at least one week, two weeks, six weeks, eight weeks, ten weeks, twelve weeks, fourteen weeks, sixteen weeks, eighteen weeks, twenty weeks, twenty two weeks twenty four weeks, seven months, eight months, ten months, twelve months, fourteen months, sixteen months, eighteen months, twenty months, twenty two months or twenty four months following the first injection.

The triggering event, detection of which during the time interval after the first injection may trigger administration of the second injection, may be a sustained reduction, or sustained insignificant change, in the patient's eGFR relative to his or her baseline eGFR, e.g., as discussed earlier herein. The triggering event, detection of which during the time interval after the first injection that may trigger administration of the second injection, may be a sustained increase, or sustained insignificant change, in the patient's uACR relative to his or her baseline uACR, e.g., as discussed earlier herein. A sustained reduction, or sustained insignificant change, in eGFR relative to baseline or a sustained increase, or sustained insignificant change, in uACR relative to baseline may be one that is maintained over a period of time, e.g. maintained for at least 7 days, at least 14 days, at least 21 days, at least 28 days, at least 30 days, at least 45 days, at least 60 days, at least 75 days, or at least 90 days.

The triggering event, thus, if it is a sustained reduction, or sustained insignificant change, in patient eGFR, may be a reduction, or insignificant change, in the patient's eGFR that is maintained for at least about 7 days, 14 days, 21 days, 28 days, 30 days, 45 days, 60 days, 75 days or 90 days. By way of example, if the triggering event is a sustained reduction in patient eGFR of at least about 5% relative to patient baseline eGFR, then the triggering event may be detected as a reduction in the patient's baseline eGFR by at least about 5% that is maintained for at least about 7 days, 14 days, 21 days, 28 days, 30 days, 45 days, 60 days, 75 days or 90 days. If the triggering event is a sustained reduction in patient eGFR of at least about 25% relative to patient baseline eGFR, then the triggering event may be detected as a reduction in the patient's baseline eGFR by at least about 25% that is maintained for at least about 7 days, 14 days, 21 days, 28 days, 30 days, 45 days, 60 days, 75 days or 90 days. It will be understood that such a sustained reduction in eGFR, (e.g., of at least 5%), for any time period, (e.g., at least about 7 days, 14 days, 21 days, 28 days, 30 days, 45 days, 60 days, 75 days or 90 days), does not require that the reduction in the patient's eGFR relative to baseline, once reduced by a certain percentage (e.g., at least about 5%), be maintained at that certain percentage reduction, or to continue to be further reduced, over time (e.g., at least about 7 days, 14 days, 21 days, 28 days, 30 days, 45 days, 60 days, 75 days or 90 days). Rather, it will be understood that the sustained reduction in eGFR, (e.g., of at least 5%), for any time period, (e.g., at least about 7 days, 14 days, 21 days, 28 days, 30 days, 45 days, 60 days, 75 days or 90 days) may refer to patient's eGFR, once it is reduced by the certain percentage (e.g., at least about 5%), remaining at least reduced by that certain percentage reduction over time (e.g., at least about 7 days, 14 days, 21 days, 28 days, 30 days, 45 days, 60 days, 75 days or 90 days). A sustained reduction, or insignificant change, in patient eGFR relative to baseline eGFR may be detected by determining patient eGFR at multiple time points (e.g., at least one, at least two, at least three, at least four, at least five, at least six, at least seven, at least eight, at least nine, at least ten time points) following administration of the first injection with the therapeutic, SRC-containing, composition. The multiple time points following administrations may occur with any frequency and need not occur at regular intervals.

The triggering event, if it is a sustained increase, or sustained insignificant change, in patient uACR, may be an increase, or insignificant change, in the patient's uACR that is detected as being maintained for at least about 7 days, 14 days, 21 days, 28 days, 30 days, 45 days, 60 days, 75 days or 90 days. By way of example, if the triggering event is a sustained increase in patient uACR by at least 30 mg/g and at least about 20% relative to baseline, then the triggering event may be detected as an increase in the patient's baseline uACR of at least 30 mg/g and at least 20% that is maintained for at least about 7 days, 14 days, 21 days, 28 days, 30 days, 45 days, 60 days, 75 days, or 90 days. If the triggering event is a sustained increase in patient uACR by at least about 20% relative to baseline, then the triggering event may be detected as an increase in the patient's baseline uACR of at least about 20% that is maintained for at least about 7 days, 14 days, 21 days, 28 days, 30 days, 45 days, 60 days, 75 days, or 90 days. It will be understood that a sustained increase in uACR, (e.g., by at least 30 mg/g and at least 20%), for any time period, (e.g., at least about 7 days, 14 days, 21 days, 28 days, 30 days, 45 days, 60 days, 75 days or 90 days), does not require that the increase in the patient's uACR relative to baseline, once increased by 30 mg/g and a certain percentage (e.g., at least about 20%), be maintained at the increase of 30 mg/g and that certain percentage, or that it continue to be further increased, over time (e.g., at least about 7 days, 14 days, 21 days, 28 days, 30 days, 45 days, 60 days, 75 days or 90 days). Rather, it will be understood that any sustained increase in uACR, (e.g., by at least about 7 days, 14 days, 21 days, 28 days, 30 days, 45 days, 60 days, 75 days or 90 days), for any time period, (e.g., at least about 7, 14, 30, 45, 60, 75 or 90 days) may refer to patient's uACR, once it is increased by the about 30 mg/g and the certain percentage (e.g., at least about 20%), remaining at least increased by about 30 mg/g and remain increased no less than that certain percentage (e.g., at least about 20%) over time (e.g., at least about 7, 14, 30, 45, 60, 75 or 90 days). The sustained increase, or insignificant change, in patient uACR relative to baseline uACR may be detected by determining patient uACR at multiple time points (e.g., at least one, at least two, at least three, at least four, at least five, at least six, at least seven, at least eight, at least nine, at least ten time points) following administration of the first injection with the therapeutic, SRC-containing, composition. The multiple time points following administrations may occur with any frequency and need not occur at regular intervals.

The use of about or approximately throughout, in reference to a specified value, (e.g., unit/quantity of time, cell number, patient eGFR, percentage improvement, percentage of cells expressing a marker, dose, etc.) should be understood to permit the specified value to be within a range, e.g., not be exactly as specified. For example, the use of about or approximately in reference to a specified value may permit the value to be within 3% (greater or less than), 4% (greater or less than), 5% (greater or less than), 6% (greater or less than), 7% (greater or less than) 8% (greater or less than), 9% (greater or less than) or 10% (greater or less than) of that specified.

Those skilled in the art will recognize, or be able to ascertain using no more than routine experimentation, many equivalents to the specific embodiments described herein. Such equivalents are intended to be encompassed by the appended claims.

All publications, patents and patent applications mentioned in this specification are herein incorporated by reference into the specification to the same extent as if each individual publication, patent or patent application was specifically and individually indicated to be incorporated herein by reference in their entirety.

### EXAMPLES

### Example 1—Two Doses of Autologous Renal Cell, SRC, Therapeutic Restores Kidney Function in D-CKD Patients

Introduction. Cell-based regenerative therapies may have the potential to modulate or reverse diseases and offer new singular or complimentary treatments for CKD. A study was conducted to determine whether two injections with an autologous renal cell, SRC, therapeutic could improve kidney function in patients with Type 2 D-CKD.

Methods/Study population: D-CKD patients aged 30-80 years, with an eGFR of 20-50 mL/min/1.73 m², were eligible for enrolment in the parent study (NCT02836574). Sodium-glucose transport protein 2 (SGLT2) receptor blockade treatment was not a contraindication. Patients were managed with standard of care for comorbidities. A subset analysis of patients from the phase II parent study treated with REACT were analyzed by FACS.

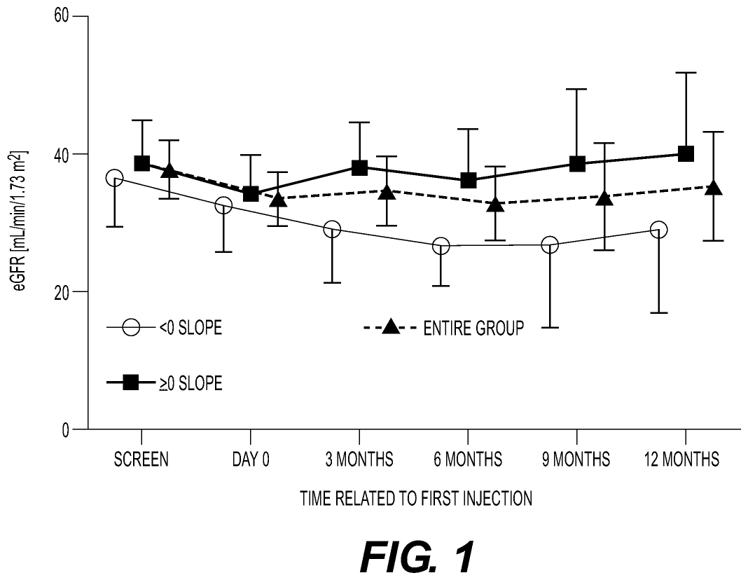

After having obtained their written informed consent, patients were enrolled at a screening visit, which occurred approximately 3 months before their first injection with the SRC-containing therapeutic. Patients with incapacitating comorbidities, except for diabetes-related complications were excluded. Patients who received first and second injections and two patients with one injection with the SRC therapeutic and who had given written informed consent for the publication of their data were included in the presented sub-analysis set shown in FIGS. 1, 4 and 5.

Methods/Data Collection: Clinical data were collected at the time of the first injection, and every 3 months for at least one year after the first injection.

Methods/Autologous Renal Cell Formulation Preparation: All patients underwent a standard percutaneous kidney biopsy. Kidney cells were isolated from their biopsies and the isolated kidney cells were expanded and treated to obtain cells for inclusion in the SRC therapeutic. The preparation of SRCs for inclusion in the SRC therapeutic have been described (e.g., in Bruce, A. T. et al., Ex vivo culture and separation of function renal cells. Methods Mol. Biol. 1001 (2013): 53; Halberstadt, C. et al., Formulation of selected renal cells for implantation into a kidney. Methods Mol. Biol. 1001 (2013): 279). After preparing SRCs, the SRCs were formulated in thermolabile hydrogel to make a fresh product, which may be referred to as REACT.

Methods/Autologous Renal Cell Formulation Administration: A first computed tomography (CT)-guided percutaneous injection of a patient's SRC therapeutic was administered approximately 3 months after the biopsy. A second percutaneous injection of the patient's SRC therapeutic was administered 3-6 months after the first injection. All day-admission procedures took place under conscious sedation protocols using intravenous midazolam and fentanyl.

Methods/Clinical and Laboratory Data: Clinical and laboratory data were collected at the time of the first SRC therapeutic injection, and every three months for one year after the first injection. Biochemical measures included electrolytes, hemoglobin, serum creatinine (sCr), bound urea nitrogen (BUN), phosphorus, calcium, potassium, bicarbonate, glycated hemoglobin (HbAlc), log-transformed intact parathyroid hormone (PTH), and log-transformed urinary albumin/sCr ratio (UACR). eGFR was estimated using the 2009 CKD-EPI formula for IDMS traceable serum sCr alone and the 2012 CKD-Epi formula for sCr and cystatin C (nephelometry with certified reference materials) (Levey A S, Stevens L A, Schmid C H, et al. A new equation to estimate glomerular filtration rate. Annals of internal medicine 2009; 150:604-612; Inker L A, Schmid C H, Tighiouart H, et al. Estimating glomerular filtration rate from serum creatinine and cystatin C. The New England journal of medicine 2012; 367:20-29). The cohort was analyzed before and after having received the first SRC therapeutic injection, establishing their eGFR slope pre-intervention. The patients were classified based on their annualized eGFR slopes (in ml/min/1.72 m²) as low responders (less than 0), moderate responders (>=0 and 2) and high responders (>2), but for analysis purposes, the moderate and high responder groups were combined and compared to low responders.

Methods/Adverse Events (AE): AEs were determined by event seriousness and intensity per reporting from the Medical Dictionary for Regulatory Activities (MedDRA) version 23.0, into Preferred Terms and System Organ Classes.

Methods/Statistics: Simple descriptive statistics were used for screening demographic and laboratory measures. Normally distributed parameters (determined via Shapiro-Wilk Normality Test) were expressed as mean and standard deviation in brackets. Not normally distributed parameters were log-transformed as those were normally distributed. Longitudinal data were compared using a linear mixed effect models (LMEM; Follmann D, Wu M. An approximate generalized linear model with random effects for informative missing data. Biometrics 1995; 51:151-168). A power calculation was not performed, as the sample size was small. For UACR, some patients had urinary creatinine and microalbumin determined in a 24-hour urine rather than a random urine sample, and here the random UACR was imputed.

Results Patients in the Data Analysis Sets: Twenty eight patients were eligible for inclusion in the trial's sub-analysis set. Of the twenty eight, two received only a single injection (one commenced on clopidogrel after the first injection and the second had uncontrolled hypertension) and six did not give consent to have their data published (two were high responders). Thus, the final number of patients who received two SRC therapeutic injections (twenty) or received one injection (two), whose data is presented in this sub-analysis data set, is twenty-two. In these twenty-two patients included in the analysis, blood pressure did not change (data not shown). The characteristics of these twenty-two patients is listed in Table 1.

TABLE 1
Patient characteristics at the time of screening

| Characteristic at the time of screening | Value |
|---|---|
| [n = 22, 15 male] | [mean ± standard deviation] |
| Age | 66.0 (±9.07) years |
| Body Mass Index | 33.9 (±5.87) kg/m² |
| Duration of Diabetes | 18.4 (±8.80) years |
| Race | 82% Caucasian, 4.5% African American, |
| | 4.5% American Indian, 9% others |
| eGFR based on 2009 CKD-EPI using sCr only | 37.3 (±8.91) mL/min/1.73 m² |
| eGFR based on 2012 CKD-EPI | 40.3 (±9.35) mL/min/1.73 m² |
| (combined sCr and Cystatin C) | |
| Log(Urinary albumin/creatinine ratio mg/g) | 5.7 (±2.00) |
| Hemoglobin | 12.4 (±1.57) g/dL |
| Serum calcium | 9.3 (±0.55) mg/dL |
| Serum phosphate | 3.7 (±0.66) mg/dL |
| Log(Intact parathyroid hormone pg/mL) | 4.1 (±0.58) |
| Potassium | 4.7 (±0.51) mmol/L |
| Bicarbonate | 20.4 (±2.70) mmol/L |
| Hemoglobin A1c | 7.0 (±1.05)% |

Impact of medications on laboratory findings were not analyzed because only three of these patients were on phosphate binders, two were on erythropoietin supplementation and four were on potassium binders.

At baseline, the patients had a mean CKD-EPI 2009 eGFR of 37.3±8.91 mL/min/1.73 m² and a mean HgbAlc of 7.0±1.05. Consistent with the expected decline of eGFR, the eGFR dropped to 33.0±8.39 mL/min/1.73 m² at the time of the first injection.

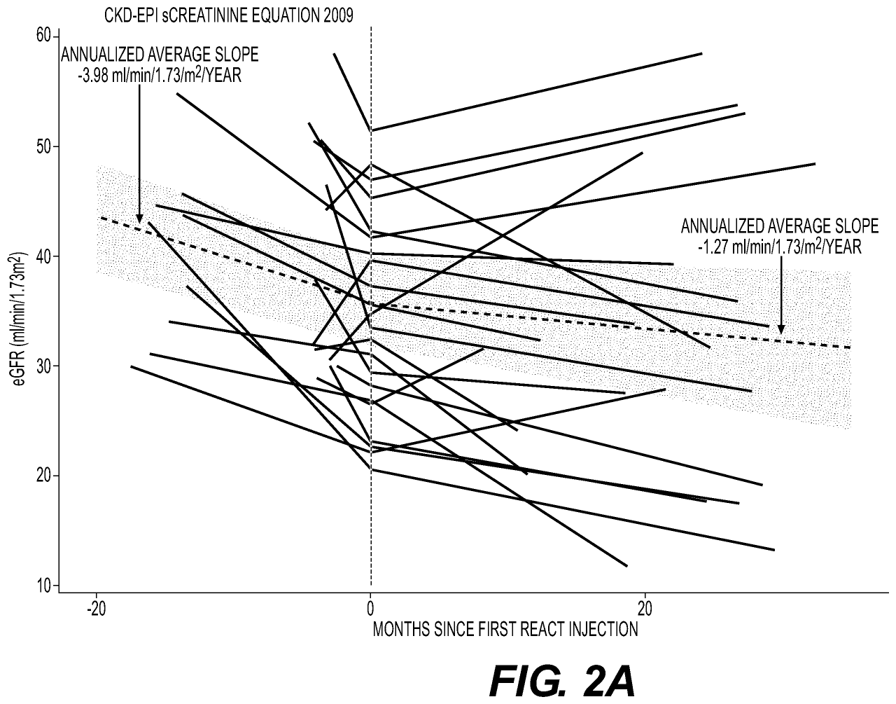

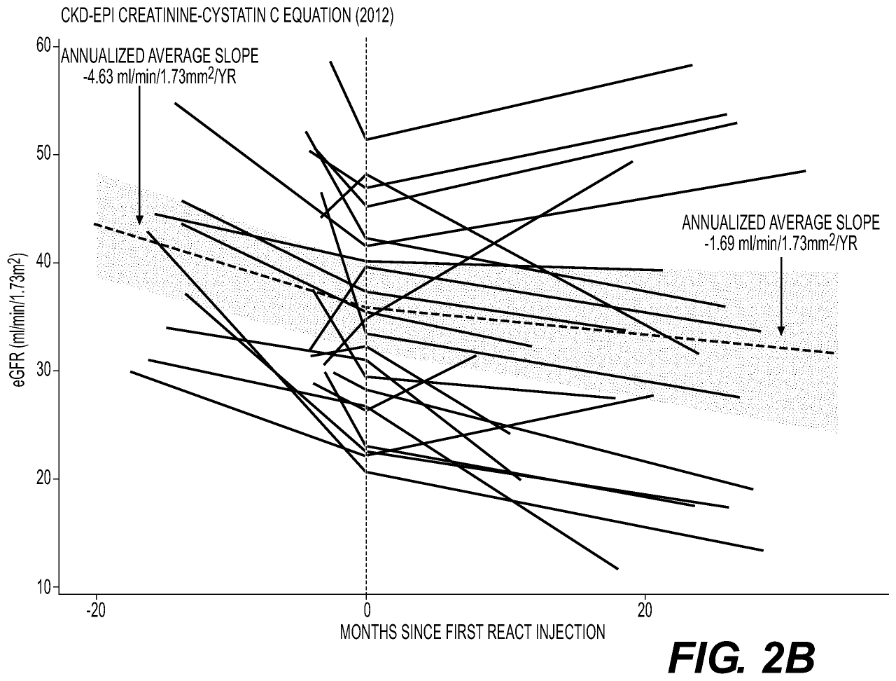

Results/Impact of Therapy on D-CKD Patient eGFR: One year following administration of the second of two SRC therapeutic injections, the group of twenty D-CKD patients in the sub-analysis set showed a stabilization of eGFR decline. See FIG. 1. Using LMEM, the annualized eGFR slope improved significantly between pre-(−4.12 mL/min/1.73 m²) and post-SRC injection (−1.23 mL/min/1.73 m²) in the full cohort (p=0.019). Using the 2009 CKD-EPI equation, based on sCr, the annualized eGFR slope was −3.98 mL/min/1.73 m² pre-injection and improved to −1.27 mL/min/1.73 m² post-injection (see FIG. 2A). Using the 2012 CKD-EPI equation, based on sCR and cystatin C, the annualized eGFR slope was −4.69 mL/min/1.73 m² pre-injection and improved to −1.69 mL/min/1.73 m² (p=0.007) post-injection (see FIG. 2B).

Patients of the sub-analysis set were observed to have one of three types of renal function response, which were considered to be clinically relevant: high responder (n=4) with a substantial improvement of eGFR and a post-treatment slope >+2, moderate responder (n=3) with a slope between zero and +2, and low responder (n=15) with a slope <0 mL/min/1.73 m². Overall, the eGFR slope became less negative in all patients, suggesting kidney function stabilization and patients with a negative slope still showed some improvement. Of note, those with a positive slope had higher eGFR at entry.

The seven patients (32%) that had a positive post-injection eGFR slope (“moderate responders” and “high responders”) tended to have a longer time, e.g., approximately one month, between the two SRC therapeutic injections (p=0.0906) and their entry eGFR slope post-first injections was 40.8±8.26 mL/min/1.73 m² (higher than that of the remaining, “low responders”, n=15 [31.5±8.11 mL/min/1.73 m², p=0.03, t-test]). These seven patients also had a log UACR of 4.5±1.99 (lower than that compared to other 15 patients (6.5±1.71, p=0.07, t-test)). Their mean SRC-containing cumulative dose was 10.4±2.84 mL, which was not significantly different from the other 15 patients (11.2±2.74 mL, p=0.5649). Other laboratory values were not different (data not shown).

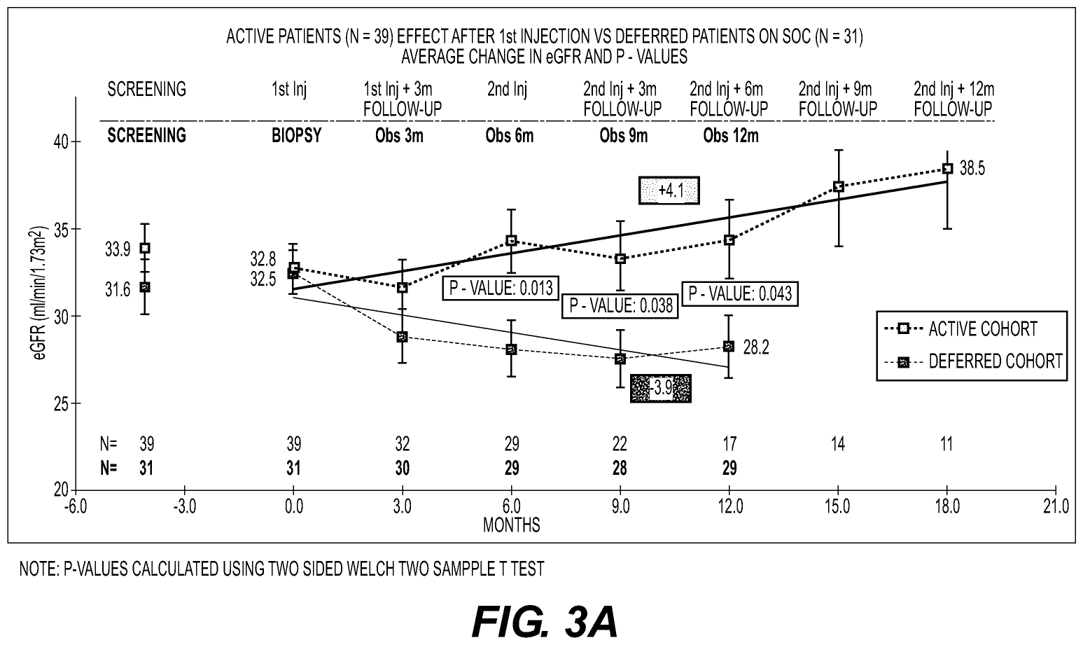

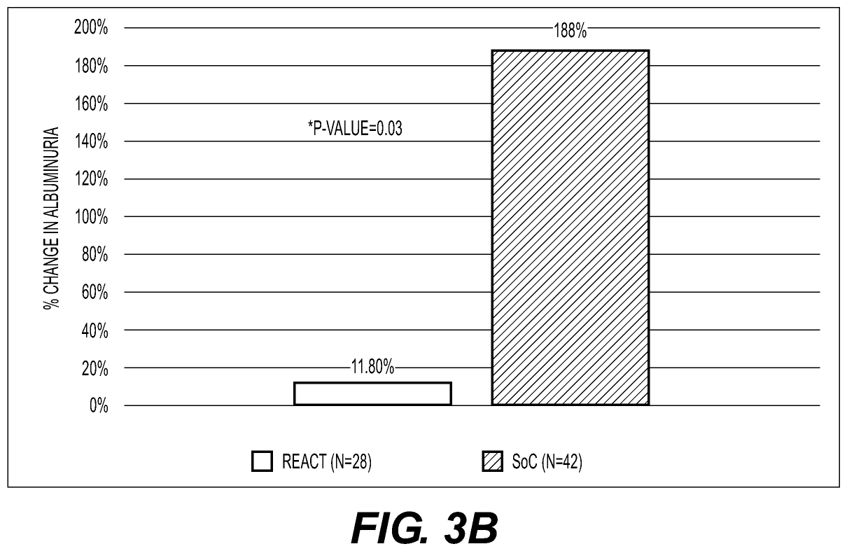

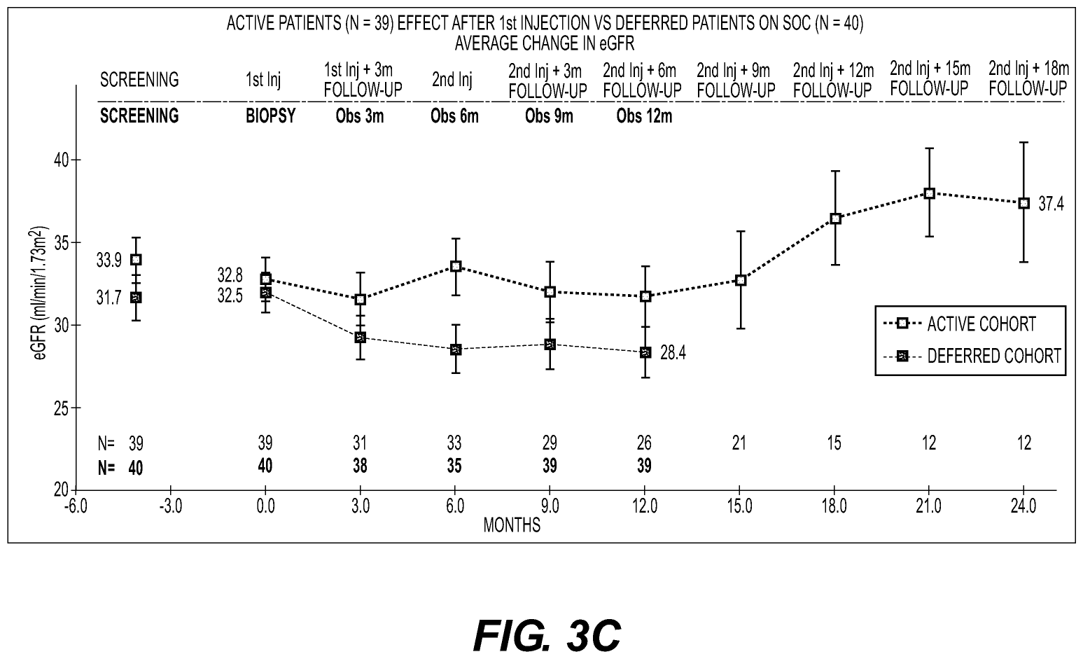

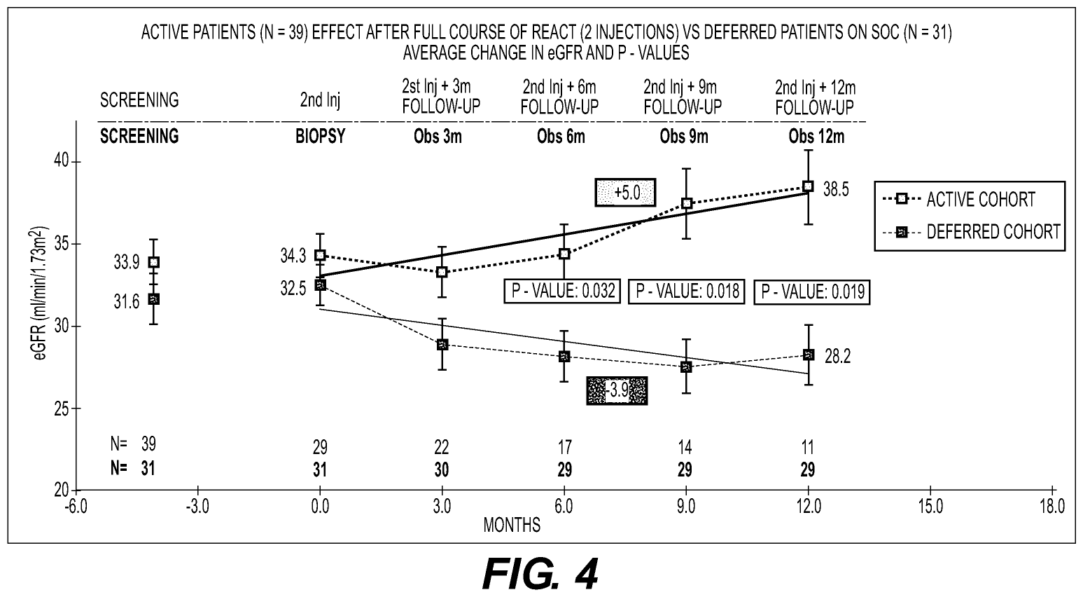

Examination of data from a larger set of patients in the trial, comparing eGFRs of those treated with the SRC therapeutic versus those treated with SOC therapy showed that SRCs have a dramatic effect on CKD patients' eGFRs. FIG. 3A, 3C and FIG. 4 show eGFR increases over time for D-CKD patients treated with two injections of the SRC therapeutic while eGFR of D-CKD patients treated with SOC therapy does not increase. Following the second SRC therapeutic injection, D-CKD patients' eGFRs consistently increased over at least one year, by 5 mL/min/1.73 m². This consistent improvement in eGFR not been observed for any therapy or therapeutic agent for D-CKD. As expected, the SOC-treated D-CKD patients' eGFR consistently declined over the same one-year period.

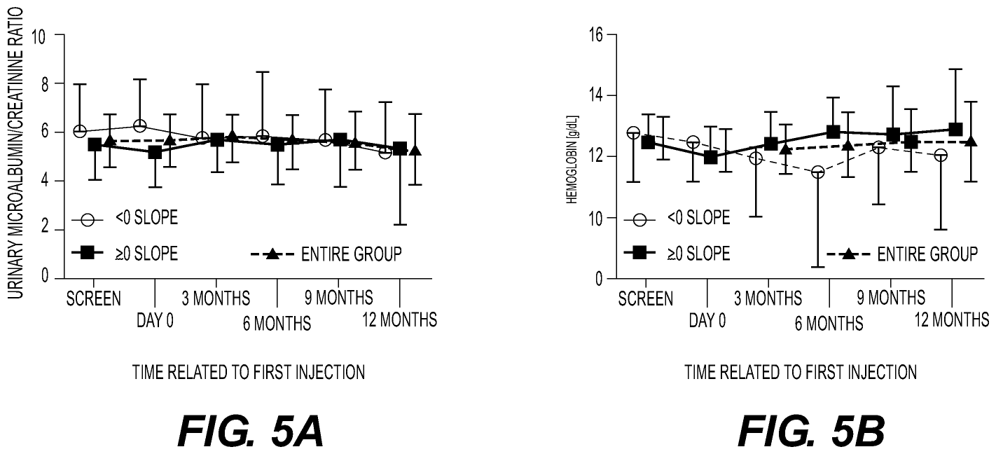

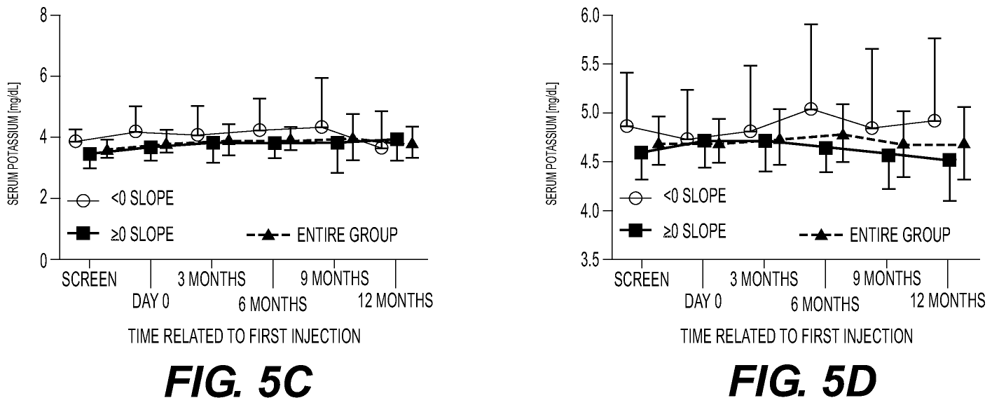

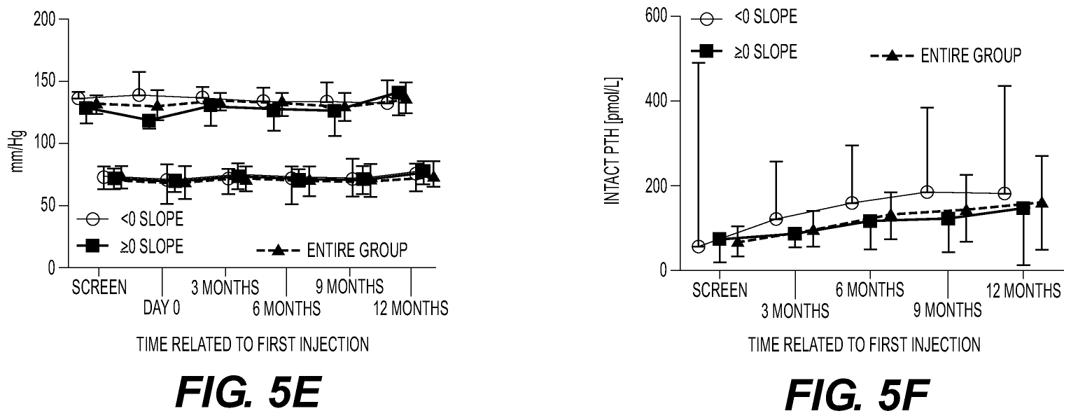

Results/Impact of Therapy on Laboratory Assessments of Kidney Function: Overall, two injections of the SRC therapeutic stabilized urine microalbumin/creatinine ratio (UAUC; FIG. 5A), haemoglobin (FIG. 5B), serum potassium (FIG. 5D), blood pressure (FIG. 5E), serum phosphate (FIG. 5C) and intact parathyroid hormone (iPTH; FIG. 5F) in all D-CKD patients—with a trend to lower potassium and lower iPTH in the moderate/high responder group.

LMEM analyses for laboratory parameters showed significantly lower annualized BUN slope from 4.88 mg/dL/year pre-injection to 0.56 mg/dL/year post-injection (p=0.045), and, again, stabilization of potassium, hemoglobin, and biomarkers of kidney osteodystrophy. See Table 2. A slight worsening of log PTH and log UACR were observed, although those changes may not be clinically relevant and could not be adjusted because of the small number of participants. HbAlc level was not a factor in eGFR recovery. Blood pressure was unchanged except in one patient who received only one REACT injection.

TABLE 2
Linear mixed effects model analysis of various clinical parameter
comparing pre- and post-injection annualized slope

| | Pre Injection | Post Injection | P |
|---|---|---|---|
| | Annualized Slope | Annualized Slope | value |
| eGFR (CKD-EPI | −3.98 ml/min/ | −1.27 ml/min/ | 0.032 |
| Creatinine 2009 | 1.73/m²/year | 1.73/m²/year | |
| Equation) | | | |
| eGFR (CKD-EPI | −4.63 ml/min/ | −1.69 ml/min/ | 0.015 |
| Creatinine-Cystatin C | 1.73/m²/year | 1.73/m²/year | |
| 2012 Equation) | | | |
| Serum Creatinine | 0.14 mg/dL/year | 0.18 mg/dL/year | 0.54 |
| Cystatin C | 0.1 mg/L/year | 0.13 mg/L/year | 0.59 |
| BUN | 4.74 mg/dL/year | 0.72 mg/dL/year | 0.07 |
| Phosphorus | 0.12 mg/dL/year | 0.15 mg/dL/year | 0.82 |
| Calcium | 0.02 mg/dL/year | −0.12 mg/dL/year | 0.10 |
| Serum Potassium | 0.02 mEq/L/year | 0.00 mEq/L/year | 0.83 |
| Serum Bicarbonate | −0.47 mEq/L/year | 0.33 mEq/L/year | 0.13 |
| Log (PTH) | 0.8 | 0.19 | 0.04 |
| Hemoglobin | −0.09 g/dL/year | −0.07 g/dL/year | 0.93 |
| Log (UACR) | −0.42 | 0.23 | 0.001 |

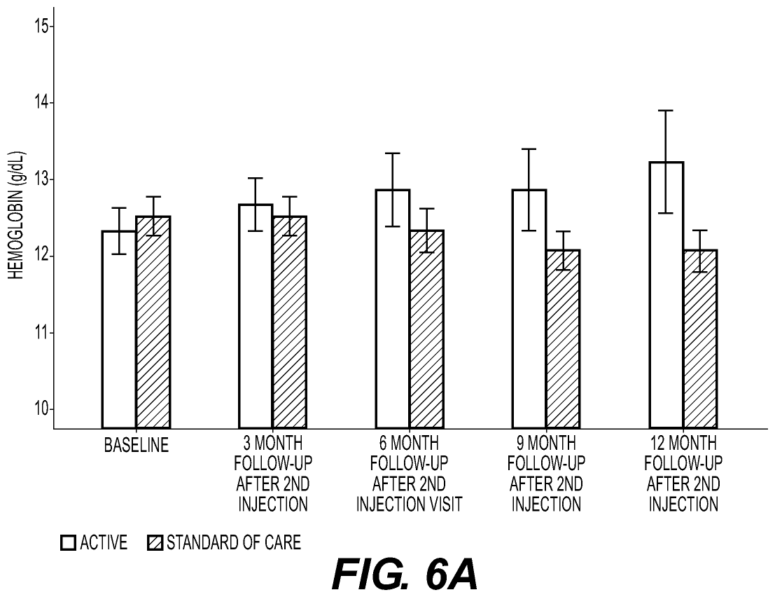

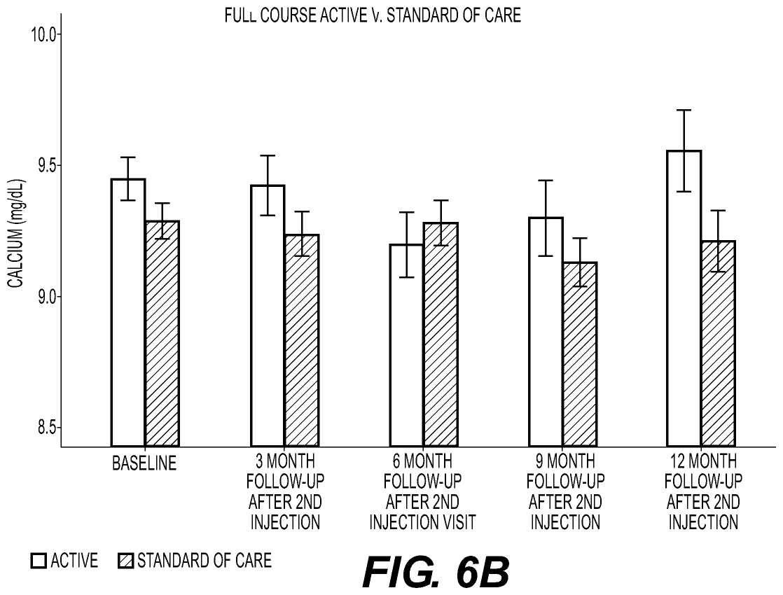

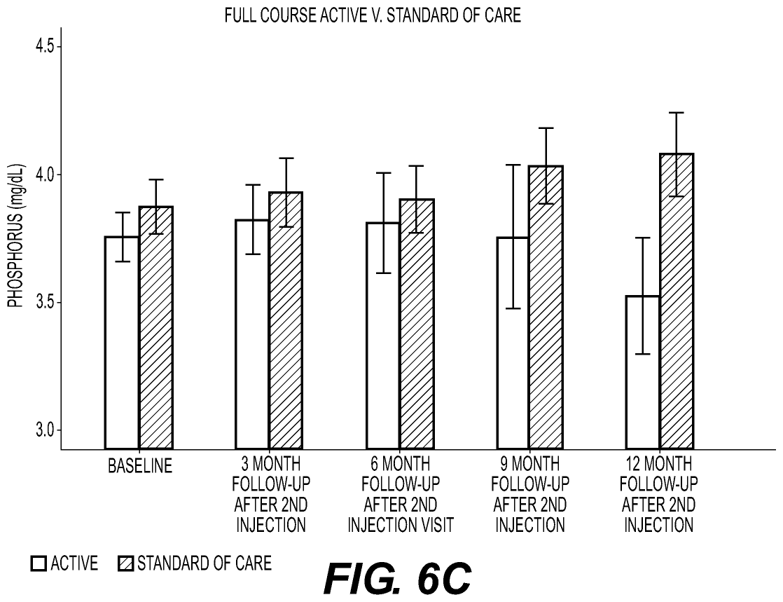

To better show the impact of administration of two injections of the SRC therapeutic on some of these parameters, a comparison was made of the results obtained from SRC-treated and SOC-treated D-CKD patients. A comparison of twenty-eight patients that received at least one dose of the SRC therapeutic to forty-two patients on SOC therapy reveal that while the patients who received the SRC therapeutic experienced a near stabilization in UACR, change of only 11.80%, the patients who received SOC therapy experienced a much greater, 188%, change. See FIG. 3B. A direct comparison of haemoglobin (FIG. 6A), serum calcium (FIG. 6B) and serum phosphorus (FIG. 6C), shows that while D-CKD patients treated with the SRC therapeutic are stable or improve, SOC-treated patients trended toward decline.

Results Serious Adverse Events: Adverse events are common in the subset population due to the co-morbidities of CKD and the metabolic syndrome. There were no serious adverse events associated with the kidney biopsy and SRC injections. A total of 61 adverse events occurred based on system organ class, with the most five common categories being cardiac, infectious, renal, respiratory and metabolic (See Table 3). Among the patients with two injections, two had SAEs of interest: one patient experienced squamous cell carcinoma of the lung causing left lung collapse/post-obstructive pneumonia and hypercalcemia and resulting in death twelve months post-second injection, but an autopsy was declined by the family. A second patient developed ESKD 11 months post-second injection and was placed on hemodialysis.

TABLE 3
Adverse Events by System Organ Class and
Preferred Terminology Based on the Medical
Dictionary for Regulatory Activities

| | Preferred |
|---|---|
| System Organ Class | terminology |
| Cardiac disorders (n = 16) | |
| Acute Myocardial Infarction | 6 |
| Atrioventricular Block Complete | 1 |
| Cardiac arrest | 2 |
| Cardiac failure acute | 2 |
| Coronary artery disease | 3 |
| Left ventricular failure | 1 |
| Myocardial infarction | 1 |
| Gastrointestinal disorders (n = 2) | |
| Diarrhea | 1 |
| Diverticulum | 1 |
| Hepatobiliary disorders (n = 1) | |
| Cholecystitis acute | 1 |
| Infections and infestations (n = 12) | |
| Cellulitis | 2 |
| Cholecystitis infective | 1 |
| Clostridium difficile infection | 1 |
| Coronavirus infection | 1 |
| Device related infection | 1 |
| Infected bite | 1 |
| Peritonitis | 1 |
| Pneumonia | 2 |
| Sepsis | 1 |
| Staphylococcal infection | 1 |
| Injury (n = 2) | |
| Fall | 1 |
| Patella fracture | 1 |
| Metabolism and nutrition disorders (n = 5) | |
| Dehydration | 1 |
| Hypercalcemia | 1 |
| Hyperkalemia | 3 |
| Musculoskeletal disorders (n = 1) | |
| Meniscal degeneration | 1 |
| Neoplasms benign/malignant (n = 1) | |
| Squamous cell carcinoma of lung | 1 |
| Nervous system disorders (2) | |
| Cerebrovascular Accident | 1 |
| Syncope | 1 |
| Psychiatric disorders (1) | |
| Hallucination, visual | 1 |
| Renal and urinary disorders (9) | |
| Acute kidney injury | 8 |
| End stage renal disease | 1 |
| Respiratory, thoracic and mediastinal | |
| disorders (6) | |
| Acute respiratory failure | 1 |
| Chronic obstructive pulmonary disease | 1 |
| Pneumothorax | 1 |
| Respiratory distress | 1 |
| Respiratory failure | 2 |
| Vascular disorders (3) | |
| Aortic stenosis | 1 |
| Deep vein thrombosis | 1 |
| Hematoma | 1 |
| Total | 61 |

Results/Summary: Two injections of the SRC therapeutic successfully improved kidney function in adults with Type 2 Diabetic Kidney Disease. All patients saw their eGFR slope improve, with forty percent demonstrating an improvement, e.g., increase, in eGFR (moderate/high responders). No other therapeutic agent has been observed to consistently halt decline in, or increase, D-CKD patients' eGFR over at least a year.

Administration of the two injections also stabilized the patients' laboratory parameters (potassium, bicarbonate, calcium, phosphate, intact PTH and haemoglobin). This, too, is noteworthy as it corroborates that the primary, filtration, function of the diseased kidney can be restored by two administrations of the SRC therapeutic. It also demonstrates that by administering the two doses, a number of renal functions are restored to the diseased kidney that thus may also address comorbidities, e.g. anaemia, associated with D-CKD.

### Example 2—Two Doses of Autologous Renal Cell, SRC, Therapeutic Restores Kidney Function in Late Stage 4 D-CKD Patients

Introduction. For D-CKD patients with an eGFR of <20 ml/min/1.72 m², treatment options do not halt the progression to ESKD. In fact, management options are often considered futile, given the accelerated loss of kidney function at this advanced CKD stage. A single-arm trial was performed in which two injections of the SRC therapeutic were administered to 30-65-year-old patients with late Stage 4 diabetic kidney disease. The trial revealed that even in a population with very high-risk for dialysis, improvement in kidney function, e.g., reduced rate of decline in eGFR, can be achieved.

Methods/Trial Design. This trial was an expansion of the trial described in Example 1 (clinical trial identifier NCT02836574). Briefly, the trial was designed such that within 60 days of the first screening assessment, a kidney biopsy was performed to obtain patient material for manufacture of his/her SRC therapeutic. Following manufacture, a first injection of the patient's SRC therapeutic was administered. A second injection was administered 3 to 6 months from the first injection. Patients were followed up for 24-months after the second injection.

Methods/Patients. Adults who had established type 2 diabetes and late stage 4 DKD with an eGFR of 14-20 ml/min/1.73 m² were eligible for inclusion in the trial. Inclusion and exclusion criteria were assessed at the screening visit, prior to renal biopsy and before each injection with the SRC therapeutic. Patients who satisfied eligibility criteria and provided written IRB-approved informed consent underwent a full physical exam, electrocardiogram, and laboratory assessments (hematology, full chemistry panel, Cystatin C, pregnancy test-females, and urine microalbumin-to-creatinine ratio).

During screening visits, patients underwent qualification physical exams, final review of inclusion/exclusion criteria and a renal scintigraphy study (split kidney function scan) to measure what percentage each kidney contributes to total kidney function. An ultrasound confirmed the presence of two kidneys with anatomic features and adequacy for percutaneous biopsy. Kidney size and volume were determined with magnetic resonance imaging or CT scan, to calculate the SRC product dose to be injected.

Methods/Renal Biopsy and Therapeutic Composition Preparation. Within 60 days of the first screening assessment, each patient underwent an image-guided percutaneous kidney biopsy of either kidney. To provide sufficient material for the manufacture of the SRC-containing therapeutic, 2-4 kidney biopsy cores were collected. The kidney biopsy was performed as an out-patient procedure with local standard of care, conscious sedation and observation. Hemorrhage post-injection of the therapeutic was determined by post-procedure ultrasound and hemoglobin measurement per trial protocol. The kidney biopsy cores were shipped overnight in specialized transport medium to ProKidney in North Carolina, USA.

Preparation of SRCs from renal biopsies has been described (e.g., in Bruce, A. T. et al., Ex vivo culture and separation of function renal cells. Methods Mol. Biol. 1001 (2013): 53; Halberstadt, C. et al., Formulation of selected renal cells for implantation into a kidney. Methods Mol. Biol. 1001 (2013): 279). After the SRCs were prepared, they were formulated in a thermolabile gelatin-based hydrogel concentration of 100×10⁶ cells/mL.

Methods/SRC-containing therapeutic administration. SRCs were percutaneously injected into the renal cortex of the same kidney that underwent the renal biopsy with CT image guidance. The dose administered was 3×10⁶ cells/g estimated kidney weight, determined by renal volume analysis, with a dose volume range of 3-8 milliliters. All injections were performed as outpatient procedures under conscious sedation, using standard fentanyl and midazolam doses as needed (Stavas J, Gerber D, Coca S G, Silva A L, et al. Novel Renal Autologous Cell Therapy for Type 2 Diabetes Mellitus Chronic Diabetic Kidney Disease: Clinical Trial Design. Am J Nephrol. 2022 Jan. 14:1-9; Yu H, Sonntag P D, Bream P R, et al. Safety and Feasibility of a Novel Percutaneous Locoregional Injection Technique of Renal Cellular Therapy for Chronic Kidney Disease of Diabetes. Kidney Int. Rep. 2021 Jun. 19; 6 (9): 2486-2490). A post-injection renal ultrasound and hemoglobin and hematocrit levels were obtained during the recovery period and at 24-hours, to detect adverse bleeding events. Each patient was administered two injections, 3-6 months apart, in the same kidney where the biopsy was obtained.

Methods/Follow-Up Evaluations: Patients underwent evaluations on days 1, 7, 14, 28 (±3 days) and months 2, 3, 4 and 5 (±7 days) after the first injection with the SRC therapeutic, and on days 1, 7, 14, 28 (±3 days) and months 2 and 3 (±7 days) after the second injection. Following the second injection, assessments of safety and efficacy through 6, 9, 12, 15-, 18-, 21-, and 24-months post-treatment took place. Non-contrast MRIs were performed during screening and at the end of study, to assess morphologic changes. Renal scintigraphy evaluated split function prior to the 1^{st} and 2^{nd} injections, at 6- and 12-months post 2^{nd} injection and at the end of the study.

Methods/Disease Progression Classification. Patients were classified according to those who had stable renal function and those who required renal replacement therapy. Before administration of the injections with the SRC therapeutic, risk of dialysis was calculated with the Kidney Failure Risk Equation (8 Variable), based on Tangri et al., (Tangri N, Stevens L, Griffith J, et al. A predictive model for progression of chronic kidney disease to kidney failure. JAMA. 2011; 305 (15): 1553-1559; Kidney Risk Failure Equation (8 Variable). https://qxmd.com/calculate/calculator_125/kidney-failure-risk-equation-8-variable; accessed 21 Feb. 2022).

Methods/Adverse Events. Biopsy-, procedure-, and cell-related adverse events were monitored and recorded based on seriousness and intensity, per the Medical Dictionary for Regulatory Activities version 23.0, into Preferred Terms and System Organ Classes. In addition, all other remote adverse events were recorded.

Methods/Statistical Analysis Methods. All analyses were descriptive in nature to characterize the patients' demographic, clinical and laboratory information. Categorical variables were summarized by frequency count and percentages. Normally distributed continuous variables were summarized by presenting the mean and standard deviation; otherwise, the median was reported for not normally distributed continuous variables. The annualized eGFR slope was calculated using a longitudinal linear mixed effect models (LMEM) with a correlated random intercept and slope (Vonesh E, Tighiouart H, Ying J et al. Mixed-effects models for slope-based endpoints in clinical trials of chronic kidney disease. Stat Med. 2019; 38:4218-4239). UACR was reported as random UACR, but if random UACR was missing and 24 hr UACR was collected, then it was imputed. Baseline was defined as the last measurement before administration of the patient's first SRC therapeutic injection.

Results Patients. Five men and 5 women were enrolled in the study. The 10 patients underwent 19 SRC-containing therapeutic injections (1 participant received only one injection). All patient core biopsy samples produced an adequate amount of material for preparing the SRCs for inclusion in the therapeutic. Patient baseline characteristics were as follows: 3 Hispanic or Latino, 7 Non-Hispanic, 7 White. Mean and standard deviations (SD) were for age 58.9 (5.22) years; BMI 35.8 (8.2); age at diagnosis 58.9 (5.22) years, and time with type 2 diabetes mellitus of 13.7 years (range 5-27). See Table 4 for further cohort characteristics including baseline laboratory results and concomitant medications.

TABLE 4
Patient Baseline Characteristics and Medications (N = 10)

| | Mean (SD, %) | |
|---|---|---|
| Patient Information | | |
| Age | 58.9 | (5.22) |
| Gender (Male) | 5 | (50%) |
| Gender (Female) | 5 | (50%) |
| Hispanic or Latino | 3 | (30%) |
| Non-Hispanic or Latino | 7 | (70%) |
| Asian | 1 | (10%) |
| White | 7 | (70%) |
| Other race | 2 | (20%) |
| Body Mass Index | 35.8 | (8.2) |
| Laboratory | | |
| eGFR CKD-EPI (sCr) | 15.5 | (2.72) |
| eGFR CKD-EPI (sCr + Cys-C) | 17.7 | (3.67) |
| Serum Creatinine (mg/dl) | 3.6 | (0.73) |
| Cystatin C (mg/L) | 2.6 | (0.52) |
| Phosphorus (mg/dL) | 4.8 | (0.89) |
| Hemoglobin (g/dL) | 10.5 | (1.12) |
| Hematocrit (%) | 32.1 | (4.08) |
| UACR random log (mg/g) | 7.9 | (1.01) |
| UACR random IQR (mg/g) | 3198 | (1733-5524) |
| Urea Nitrogen (mg/dL) | 60.8 | (16.4) |
| Serum Potassium (mEq/L) | 5.2 | (0.49) |
| Serum Sodium (mEq/L) | 140.4 | (2.8) |
| Serum Bicarbonate (mEq/L) | 19.2 | (2.49) |
| HbA1c (%) | 7.7 | (1.59) |
| Intact PTH (pg/mL) | 5.2 | (0.83) |
| Calcium (mg/dL) | 9.0 | (0.62) |
| Albumin (mg/dL) | 4.0 | (0.42) |
| Concomitant Medications | n (%) | |
| ACEi | 2 | (20) |
| ARBs | 6 | (60) |
| Beta Blockers | 6 | (60) |
| Alpha 2 Agonist | 0 | (0) |
| Diuretics | 10 | (100) |
| Platelet aggregate inhibitors | 6 | (60) |
| Potassium lowering agents | 3 | (30) |
| Polystyrene sulfonate | 0 | (0) |
| Potassium-binding agents | 1 | (10) |
| Glucose lower therapies | 9 | (90) |
| Insulin | 7 | (70) |
| Metformin | 0 | (0) |
| Sulfonylurea | 2 | (20) |
| DPP-4 inhibitors | 1 | (10) |
| GLP-1 agonists | 2 | (20) |
| SGLT2 inhibitors | 0 | (0) |
| TZDs | 1 | (10) |
| Meglitinides | 0 | (0) |
| Phosphate Binders | 4 | (40) |
| Antianemic Preparations | 8 | (80) |
| Iron Preparations | 5 | (50) |
| Erythropoietins | 7 | (70) |
| Other Blood Products | 0 | (0) |
| Vitamin B12 and Folic Acid | 2 | (20) |
| Bicarbonate Supplement | 5 | (50) |
| 1-25 OH Vitamin D | 1 | (10) |
| Other Antihypertensives | 5 | (50) |

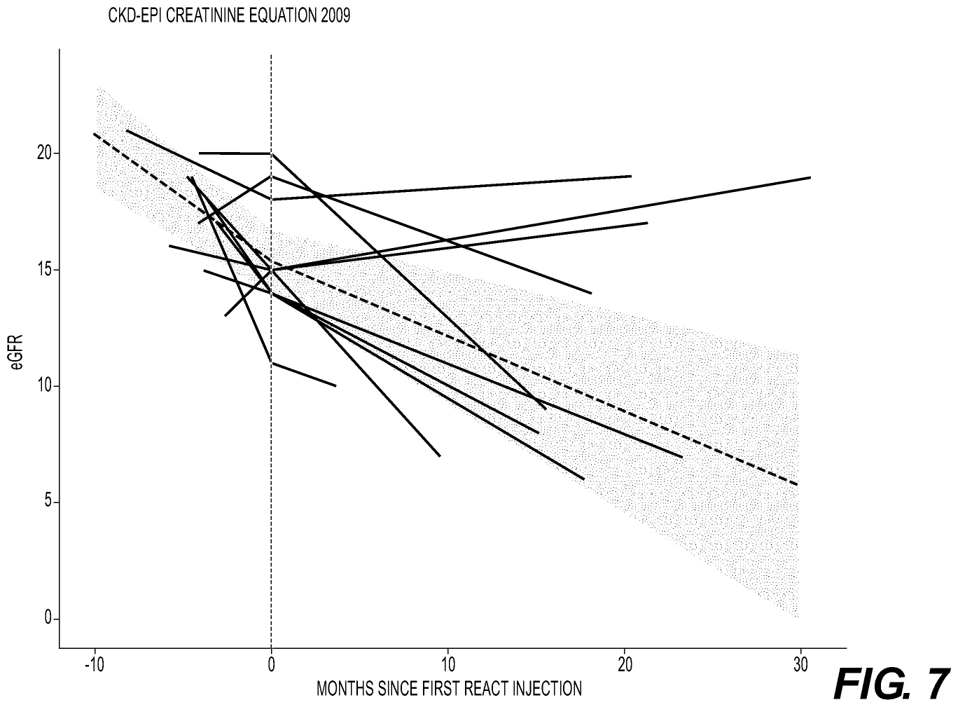

Results Improvement in Patient Kidney Function. For the study cohort, eGFR slope changed from −6.5 ml/min/1.73 m² per year pre-injection to −3.9 ml/min/1.73 m² per year post-second injection with the SRC-containing therapeutic. See FIG. 7. For individual patients in the study cohort, a comparison of pre- and post-injection slopes indicated that six of the ten had improved eGFRs, (e.g., slowed progression (stabilization)), while four of the ten had eGFR progression.

At 13.5 months post-SRC-containing injections, three patients had not begun dialysis; two were fifteen months post second injection (twenty-one months total follow up) however, the third patient died after the second injection from diabetes-related myocardial infarction without dialysis; this patient was considered lost due to an administrative error. An additional patient died after second injection due to COVID-19 infection. This patient died one day after a first episode of hemodialysis during hospitalization for COVID-19 and was included in the dialysis count despite the singular dialysis session.

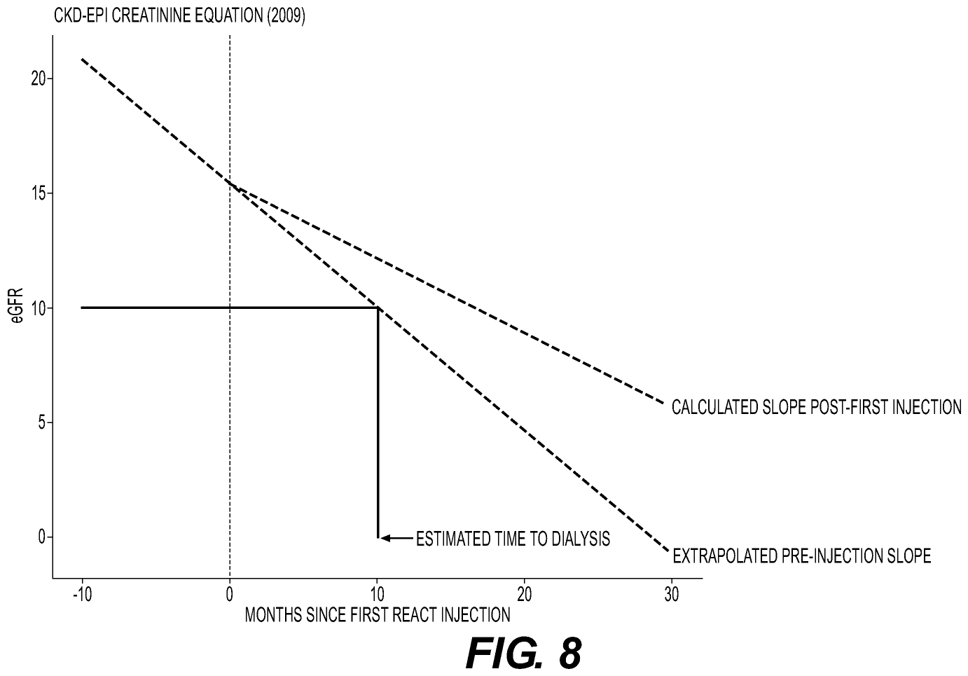

Seven patients required renal replacement therapy at a median time of 16.2 months (range 6-28 months) with eGFRs of 6-16 ml/min/1.73 m², at the time of initial dialysis. One patient with a screening eGFR of 19 ml/min/1.73 m² had rapid deterioration and heavy proteinuria and commenced dialysis 6 months after the first injection. To date, all remaining participants had received two injections. A straight-line extrapolation of the pre-injection eGFR slope (−6.5 ml/min/1.73 m²) from the time of first injection, approximates that the time to dialysis would have been 10.1 months, had the patients not been treated with the SRC therapeutic, assuming dialysis initiation at an eGFR of about 10 ml/min/1.73 m². See FIG. 8. This approximated time to dialysis is in agreement with a Chronic Renal Insufficiency Cohort (CRIC) study. In the study, the median time spent in each CKD Stage, from 3a to 5, was evaluated. Stage 5, which corresponds to the baseline eGFR of 15.5 ml/min/1.7 m² in this study, had a median time of 0.8 years. This agreement in approximated time to dialysis, e.g., about 10 months for CKD stage 5 patients, confirms the suggestion that the patients in this study fared better than if they had not received the SRC-containing therapeutic, regardless of their high co-morbidities (Ku et al., Clin. J. Am. Soc. Nephrol. 13 (2018): 693-701).

The two patients who had not received dialysis 15 months after the second of the two SRC-containing injections exhibited eGFR slope stability. These two patients, having stabilized CKD at stage 4 and lower predictive scores (65% and 75%), had lower albuminuria (UACRs 407 mg/g and 719 mg/g) compared to the dialysis group (UACRs range 1487-10889 mg/g).

Time to dialysis for each patient is provided in Table 5. Table 5 also provides the calculated risk of dialysis with the Kidney Failure Risk Equation (8 Variable), which was performed before first injection. Seven patients had a predictive score >91% for reaching ESKD within 5 years.

TABLE 5
Patient Time to Dialysis

| | 5-year risk | Time to Dialysis (months) |
|---|---|---|
| Patient | ESKD^{a} | Post-REACT injections |
| 1 | 100% | 6.0 |
| 2 | 99.9% | 16.2 |
| 3 | 99.8% | 27.9 |
| 4 | 99.5% | 20.3 |
| 5 | 99.2% | 6.1 |
| 6 | 98% | 19.4 (death)^{b} |
| 7 | 91.9% | 13.2 |
| 8 | 91% | 10.1 (death)^{c} |
| 9 | 75% | Stable CKD 4 |
| 10 | 65% | Stable CKD 4 |

^{a}Kidney Failure Risk Equation prediction compared to post-treatment time to dialysis or death.
^{b}COVID-19 related death with one episode of hemodialysis one day prior to death.
^{c}Myocardial infarction related death and not on dialysis at time of death.

Results/Adverse Events. Of all 10 single renal biopsies and 19 percutaneous injections of the SRC-containing therapeutic product, there were two instances of renal hematomas (one post-biopsy and one post-injection) that did not require blood transfusions or interventions. One patient developed a post-biopsy arteriovenous fistula that spontaneously resolved in less than 7 days. The most common system organ class side effects were in the renal and urinary disorders category (n=7) and infectious class, with two COVID-19 infections and one non-COVID-19 pneumonia. See Table 6 for all serious adverse events.

TABLE 6
Serious Adverse Events

| System Organ Class | Number |
|---|---|
| Renal and urinary disorders | 7 |
| PT: Acute kidney injury | 2 |
| PT: Chronic kidney disease | 1 |
| PT: End stage renal disease | 2 |
| PT: Renal hematoma | 2 |
| Infections and infestations | 5 |
| PT: Cellulitis | 1 |
| PT: COVID-19 | 2 |
| PT: COVID-19 pneumonia | 1 |
| PT: Pneumonia | 1 |
| Blood & lymphatic system | 2 |
| PT: Anemia | 2 |
| Respiratory & thoracic | 2 |
| PT: Acute respiratory failure | 2 |
| Vascular disorders | 2 |
| Renal arteriovenous fistula | 1 |
| Hypertensive emergency | 1 |
| General conditions | 1 |
| PT: Fatigue | 1 |
| Metabolism & nutrition | 1 |
| PT: Diabetic complication | 1 |
| Nervous system disorders | 1 |
| PT: Cerebrovascular accident | 1 |

Results/Summary. Administration of two injections of the SRC-containing therapeutic successfully improved kidney function and stabilized disease progression in a high risk, e.g., late stage 4 D-CKD, patient population. Successful improvement in kidney function in these high risk patients was able to delay ESRD and requirement for dialysis.

1. A method of improving kidney function to a human patient having diabetic chronic kidney disease (D-CKD), comprising:
administering first and second injections of a therapeutic composition comprising a selected renal cell (SRC) population into the renal cortex of at least one kidney of the human patient,
wherein the first and second injections are administered between approximately 3 and 12 months apart,
wherein the therapeutic composition comprises about 1.0-9.0×10⁶ cells of the SRC population per gram estimated kidney weight,
wherein the SRC population comprises bioactive renal cells prepared from cultured renal cells of a kidney biopsy of the patient; and
thereby improving kidney function to the human patient.

2. The method of claim 1, wherein the improving kidney function comprises:
increasing estimated glomerular filtration rate (eGFR) of the patient, and/or
stabilizing or reducing urine albumin-creatinine ratio (uACR) of the patient.

3. The method of claim 1, wherein the improving kidney function comprises restoring regulation of one or more of:
erythropoiesis by the kidney,
blood or serum potassium level by the kidney,
blood or serum phosphorus level by the kidney, or
blood or serum calcium level by the kidney.

4. The method of claim 2, wherein the improving kidney function comprises increasing eGFR of the patient.

5. The method of claim 4, wherein the increasing eGFR of the human patient comprises increasing the patient eGFR by about 1-10 ml/min/1.73 m² from a first to a second time point,
wherein the first time point is within approximately one month of the administering the first injection; and
wherein the second time point is approximately one year following the administering the first injection.

6. The method of claim 4, wherein the increasing eGFR of the human patient comprises increasing the patient eGFR by about 1-10 ml/min/1.73 m² from a first to a second time point,
wherein the first time point is within approximately one month of the administering the second injection; and
wherein the second time point is approximately one year following the administering the second injection.

7. The method of claim 5, wherein the increasing the patient eGFR is by about 1-5 ml/min/1.73 m².

8. The method of claim 6, wherein the increasing the patient eGFR is by about 1-5 ml/min/1.73 m².

9. The method of claim 5, wherein the increasing patient eGFR is by about 1-3 ml/min/1.73 m².

10. The method of claim 6, wherein the increasing patient eGFR is by about 1-3 ml/min/1.73 m².

11. The method of claim 5, wherein the increasing patient eGFR is by about 3-5 ml/min/1.73 m².

12. The method of claim 6, wherein the increasing patient eGFR is by about 3-5 ml/min/1.73 m².

13. The method of claim 4, wherein the increasing eGFR of the human patient comprises increasing the patient eGFR by at least about 2 ml/min/1.73 m², or at least about 3 ml/min/1.73 m², or at least about 4 ml/min/1.73 m², or at least about 5 ml/min/1.73 m² from a first to a second time point,
wherein the first time point is within approximately one month of the administering the first or second injection; and
wherein the second time point is approximately one year following the administering the first or second injection, respectively.

14. The method of claim 2, wherein the improving kidney function comprises stabilizing or reducing uACR of the patient.

15. The method of claim 14, wherein the improving kidney function comprises stabilizing uACR of the patient.

16. The method of claim 15, wherein the stabilizing uACR comprises maintaining uACR of the patient to within about 20% from a first time point to a second time point,
wherein the first time point is within approximately 1 month of the administering the first or second injection, and
wherein the second time point is approximately one year following the administering the first or second injection, respectively.

17. The method of claim 3, wherein the improving kidney function comprises restoring regulation of erythropoiesis by the kidney.

18. The method of claim 17, wherein the restoring regulation of erythropoiesis by the kidney comprises:
stabilizing, or returning, blood hemoglobin levels of the patient to within normal range.

19. The method of claim 3, wherein the improving kidney function comprises restoring regulation of blood or serum potassium level by the kidney.

20. The method of claim 3, wherein the improving kidney function comprises restoring regulation of blood or serum phosphate level by the kidney.

21. The method of claim 3, wherein the improving kidney function comprises restoring regulation of blood or serum calcium level by the kidney.

22. The method of claim 1, wherein the improving kidney function comprises increasing blood filtration by the kidney.

23. The method of any of claims 17-22, wherein the kidney function of the patient improves from a first time point to a second time point,
wherein the first time point is within approximately one month of the administering the first or second injection; and
wherein the second time point is approximately one year following the administering the first or second injection, respectively.

24. The method of any preceding claim, wherein improving kidney function of the human patient further comprises preventing renal replacement therapy of the patient.

25. The method of claim 24, wherein the renal replacement therapy comprises:
dialysis, or
kidney transplant.

26. The method of any of claims 1-25, wherein the human patient comprises an eGFR of greater than approximately 20 ml/min/1.73 m².

27. The method of claim 1, wherein the human patient comprises an eGFR of less than approximately 20 ml/min/1.73 m².

28. The method of claim 27, wherein the improving kidney function comprises reducing rate of decline in eGFR.

29. The method of claim 28, wherein the reducing rate of decline in eGFR is by at least about 25%.

30. The method of claim 29, wherein the reducing rate of decline in eGFR is by at least about 35%.

31. The method of claim 30, wherein the reducing rate of decline in eGFR is by at least about 45%.

32. The method of any of claims 27-31, wherein the improving kidney function further comprises preventing renal replacement therapy of the patient.

33. The method of claim 32, wherein the renal replacement therapy comprises dialysis or kidney transplant.

34. The method of claim 33, wherein the preventing renal replacement therapy comprises delaying dialysis, and the delaying dialysis is by at least about 4 months.

35. The method of claim 34, wherein the delaying dialysis is by at least about 6 months.

36. The method of claim 35, wherein the delaying dialysis is by at least about 1 year.

37. The method of claim 36, wherein the delaying dialysis is by at least about 2 years.

38. The method of any preceding claim, wherein the first and second injections are administered approximately 3 months apart, or approximately 6 months apart, or approximately 9 months apart, or approximately 12 months apart.

39. The method of any of claims 1-37, wherein the first and second injections are administered approximately 3-9 months apart.

40. The method of any of claims 1-37, wherein the first and second injections are administered approximately 6-12 months apart.

41. The method of any of claims 1-37, wherein the first and second injections are administered approximately 6-9 months apart.

42. The method of any preceding claim, wherein the first and second injections are percutaneous injections.

43. A method of improving kidney function to a human patient having diabetic chronic kidney disease (D-CKD), comprising:
administering a first injection of a therapeutic composition comprising a selected renal cell (SRC) population into the renal cortex of at least one kidney of the human patient, and
administering a second injection of the therapeutic composition into the renal cortex of at least one kidney of the human patient if, approximately 3 to 18 months after the first injection, a triggering event is detected,
wherein the triggering event comprises:
(a) a reduction, or insignificant change, in patient eGFR relative to patient baseline eGFR; or
(b) an increase, or insignificant change, in patient uACR relative to patient baseline uACR;
wherein the second injection is administered within approximately 30 days after detecting the triggering event,
wherein the therapeutic composition comprises about 1.0-9.0×10⁶ cells of the SRC population per gram estimated kidney weight,
wherein the SRC population comprises bioactive renal cells prepared from cultured renal cells of a kidney biopsy of the patient; and
thereby improving kidney function to the human patient.

44. The method of claim 43, wherein the patient baseline eGFR or uACR is the patient eGFR or uACR, respectively, at a time within between approximately 60 days prior to the first injection and the first injection.

45. The method of claim 44, wherein the patient baseline eGFR or uACR is the patient eGFR or uACR, respectively, at a time approximately 60 days prior to the first injection.

46. The method of claim 44, wherein the patient baseline eGFR or uACR is the patient eGFR or uACR, respectively, at approximately the time of the first injection.

47. The method of claim 43, wherein the patient baseline eGFR or uACR is an average baseline patient eGFR or uACR,
wherein the average baseline patient eGFR or uACR averages two patient eGFR or uACR determinations at two time points,
wherein the two time points are from between approximately 60 days prior to the first injection and the first injection.

48. The method of claim 43, wherein the patient baseline eGFR or uACR is an average baseline patient eGFR or uACR,
wherein the average baseline patient eGFR or uACR averages three patient eGFR or uACR determinations at three time points,
wherein the three time points are from between approximately 60 days prior to the first injection and the first injection.

49. The method of any of claims 43-48, wherein the triggering event is detected if the reduction, or insignificant change, in patient eGFR relative to patient baseline eGFR is determined to be sustained.

50. The method of claim 49, wherein the reduction, or insignificant change, in patient eGFR relative to patient baseline eGFR is determined to be sustained if the reduction, or insignificant change, is maintained for at least about 30 days.

51. The method of claim 50, wherein the reduction, or insignificant change, in patient eGFR relative to patient baseline eGFR is determined to be sustained if the reduction, or insignificant change, is maintained for at least about 60 days.

52. The method of claim 51, wherein the reduction, or insignificant change, in patient eGFR of relative to patient baseline eGFR is determined to be sustained if the reduction, or insignificant change, is maintained for at least about 90 days.

53. The method of any of claims 43-48, wherein the reduction, or insignificant change, in patient eGFR relative to patient baseline eGFR is at least about 5%.

54. The method of any of claims 43-48, wherein the triggering event is detected if the increase, or insignificant change, in patient uACR relative to patient baseline uACR is determined to be sustained.

55. The method of claim 54, wherein the increase, or insignificant change, in patient uACR is determined to be sustained if the increase, or insignificant change, relative to patient baseline uACR is maintained for at least 7 days.

56. The method of claim 55, wherein the increase, or insignificant change, in patient uACR is determined to be sustained if the increase, or insignificant change, relative to patient baseline uACR is maintained for at least 30 days.

57. The method of claim 56, wherein the increase, or insignificant change, in patient uACR is determined to be sustained if the increase, or insignificant change, relative to patient baseline uACR is maintained for at least 60 days.

58. The method of claim 57, wherein the increase, or insignificant change, in patient uACR is determined to be sustained if the increase, or insignificant change, relative to patient baseline uACR is maintained for at least 90 days.

59. The method of any of claim 43-48 or 54-58, wherein the increase, or insignificant change, in the patient uACR is an increase of at least about 20% and at least about 30 mg/g relative to the patient baseline uACR.

60. The method of claim 59, wherein the increase, or insignificant change, is at least about 30% and at least about 30 mg/g.

61. The method of any of claims 43-53, wherein the patient baseline eGFR is less than about 25 ml/min/1.73 m², and the triggering event comprises reduction in patient eGFR of about 5% relative to the patient baseline eGFR.

62. The method of any of claims 43-53, wherein the patient baseline eGFR is between about 25 and 35 ml/min/1.73 m², and the triggering event comprises reduction in patient eGFR of about 10% relative to the patient baseline eGFR.

63. The method of any of claims 43-53, wherein the patient baseline eGFR is between about 35 and 45 ml/min/1.73 m², and the triggering event comprises reduction in patient eGFR of about 20% relative to the patient baseline eGFR.

64. The method of any of claims 43-53, wherein the patient baseline eGFR is at least about 45 ml/min/1.73 m², and the triggering event comprises reduction in patient eGFR of about 25% relative to the patient baseline eGFR.

65. The method of any of claims 43-53, wherein the triggering event comprises the reduction in patient eGFR and the reduction is by about 25% relative to the patient baseline eGFR.

66. The method of any of claims 43-65, wherein administration of the first injection and the second injection are into the renal cortex of different kidneys of the patient.

67. The method of any of claim 43-53 or 61-66 further comprising detecting the triggering event, wherein the detecting the triggering event comprises a step of determining patient eGFR at least once following the first injection.

68. The method of claim 67, wherein the patient eGFR is determined at least twice following the first injection.

69. The method of any of claim 43-48 or 54-60, further comprising detecting the triggering event, wherein the detecting the triggering event comprises a step of determining patient uACR at least once following the first injection.

70. The method of clam 69, wherein the patient uACR is determined at least twice following the first injection.

71. The method of any of claims 43-70, wherein the improving kidney function comprises:
stabilizing or increasing estimated glomerular filtration rate (eGFR) of the patient, and/or stabilizing or reducing urine albumin-creatinine ratio (uACR) of the patient.

72. The method of any of claims 43-70, wherein the improving kidney function comprises restoring regulation of one or more of:
erythropoiesis by the kidney,
blood or serum potassium level by the kidney,
blood or serum phosphorus level by the kidney, or
blood or serum calcium level by the kidney.

73. The method of any of claims 43-72, wherein improving kidney function of the human patient further comprises preventing renal replacement therapy of the patient.

74. The method of claim 73, wherein the renal replacement therapy comprises:
dialysis, or
kidney transplant.

75. The method of any preceding claim, wherein the SRC population is prepared from the cultured renal cells having been exposed to hypoxic conditions.

76. The method of any preceding claim, wherein the cells of the SRC population exhibit a buoyant density greater than approximately 1.04 g/mL.

77. The method of any preceding claim, wherein the SRC population comprises: (i) cells that express gamma-glutamyl transpeptidase (GGT)-1; (ii) cells that express cytokeratin 18 (CK18), (iii) cells that secrete vascular endothelial growth factor (VEGF); and (iv) cells that secrete kidney injury molecule-1 (KIM-1):
wherein at least about 4.5% of the cells of the SRC population express GGT-1; and at least about 80% of the cells of the SRC population express CK18.

78. The method of any preceding claim, wherein the SRC population comprises cells that express at least one nephrogenic marker,
wherein the nephrogenic marker comprises one or more of SIX Homeobox 2 (SIX2), odd-skipped-related 1 (OSR1), LIM homeobox 1 (LHX1), rearranged during transfection (RET) or fibroblast growth factor 8 (FGF8).

79. The method of claim 78, wherein the at least one nephrogenic marker comprises LHX1, and
wherein at least about 15% of the cells of the population express LHX1.

80. The method of any of claims 1-77, wherein the SRC population comprises: (i) cells that express LHX1; (ii) cells that express nephrin; and (iii) cells that express podocin,
wherein at least about 70% of the cells of the SRC population express nephrin, at least about 80% of cells of the SRC population express podocin, and at least about 15% of cells of SRC population express LHX1.

81. The method of any preceding claim, wherein the SRC population comprises renal tubular cells, and
wherein percentage of renal tubular cells is higher in the SRC population than in the cultured renal cells.

82. The method of any preceding claim, wherein the SRC population comprises glomerular cells, and
wherein percentage of glomerular cells is higher in the SRC population than in the cultured renal cells.

83. The method of any preceding claim, wherein the SRC population comprises peritubular interstitial cells, and
wherein percentage of peritubular interstitial cells is higher in the SRC population than in the cultured renal cells.

84. The method of any preceding claim, wherein the therapeutic composition comprises approximately 3.0×10⁶ cells of the SRC population per gram estimated kidney weight.
